(19) United States
(12) Patent Application Publication
(10) Pub. No.: US 2019/0322981 A1
(43) Pub. Date: Oct. 24, 2019

(54) MEANS AND METHODS FOR THE GENERATION OF OLIGODENDROCYTES

(71) Applicant: Westfaelische Wilhelms-Universitaet Muenster, Muenster (DE)

(72) Inventors: Marc Ehrlich, Münster (DE); Tanja Kuhlmann, Münster (DE)

(21) Appl. No.: 16/315,104

(22) PCT Filed: Jul. 5, 2017
(86) PCT No.: PCT/EP2017/066729

§ 371 Date: Jan. 3, 2019

(30) Foreign Application Priority Data
Jul. 5, 2016 (LU) 93140

(51) Int. Cl. C12N 5/079

(52) U.S. Cl. CPC C12N 5/0622; C12N 2320/30; C12N 2501/01; C12N 2501/105; C12N 2501/115; C12N 2501/13; C12N 2501/135; C12N 2501/15; C12N 2501/155; C12N 2501/16; C12N 2501/395; C12N 2506/45; C12N 2510/00; C12N 2506/1307; C12N 2500/38

(57) ABSTRACT

The present invention relates to methods of generating oligodendroglial lineage cells from human cells selected from the group consisting of neural progenitor cells (NPCs), pluripotent stem cells (PSCs), induced pluripotent stem cells (iPSCs) and fibroblasts. The invention furthers relates to methods of screening for a compound promoting oligodendroglial differentiation and/or maturation, specifically to high throughput methods. In addition, the invention relates to cells obtainable by these methods and use of these cells in therapy.

### FIELD OF THE INVENTION

The present invention relates to methods of generating oligodendroglial lineage cells from human cells selected from the group consisting of neural progenitor cells (NPCs), pluripotent stem cells (PSCs), induced pluripotent stem cells (iPSCs) and fibroblasts. The invention furthers relates to methods of screening for a compound promoting oligodendroglial differentiation and/or maturation, specifically to high throughput methods. In addition, the invention relates to cells obtainable by these methods and use of these cells in therapy.

### DESCRIPTION

Oligodendroglial lineage cells (OL) play a key role in myelin related diseases including multiple sclerosis (MS), leukodystrophies as well as periventricular leukomalacia, and there is an increasing awareness of their potential role in neurodegenerative diseases (e.g. multiple system atrophy and amyotrophic lateral sclerosis) or traumatic spinal cord injury¹⁻⁶. They form and maintain the myelin sheaths that insulate axons and organize the distribution of axonal voltage-gated ion channels prerequisite for conduction of action potentials and trophic support of axons. Demyelination in MS contributes to axonal damage and disease progression⁷. Immunosuppressive or immunomodulatory therapies including complete ablation of the immune system by radiation and chemotherapy prevent new inflammatory lesions that underlie clinical relapses but do not arrest disease⁸. Therapies promoting remyelination represent a promising new treatment strategy to protect and restore axonal integrity and neurologic function⁴. The development of such therapeutics is hampered, at least in part, by the limited availability of human OL.

Thus, there is a great need for an improved availability of human OL, especially patient-specific OL. In general, provision of oligodendroglial lineage cells would permit studies to delineate mechanisms regulating repair by endogenous myelin lineage cells and/or provide a source of autologous cells for replacement therapy. Such cells would also provide new opportunities to identify pathological mechanisms underlying de- or dysmyelinating diseases.

While basic findings on the involvement of several transcription factors in oligodendroglial differentiation in the mouse model have been made, such knowledge is of limited value for the situation in man. The general fact that differences between mouse and human physiology hamper translatability of results from a mouse model to human medical research is well known. This is especially true for highly intricate regulatory networks such as those involved in developmental cell differentiation in general and relevant for myelin diseases such as multiple sclerosis (MA) and neurodegenerative diseases such as Alzheimer's disease in particular⁴⁷. Thus, the limited predictability based on insights from animal models represents a serious obstacle for providing methods for human glial cell differentiation.

Recent advances in the field of somatic cell reprogramming have enormously furthered the use and optimization of induced pluripotent stem cells (iPSCs) since the seminal studies by Yamanaka and coworkers^{48, 49}. Several human iPSC lines derived from patients suffering from different diseases have been generated, including Parkinson's disease (PD), Alzheimer's disease and schizophrenia. Moreover, gene-editing approaches have been used to correct genetic mutations on PD patient derived-iPSC, resulting in the successful reversal of pathological phenotypes^{50,51}. Thus, stem cell technologies in general and iPSCs in particular represent a promising tool for providing researchers with a supply of various cell types found in the human body. In the long term, such in vitro-differentiated cells might have an enormous impact on therapy of various diseases.

However, as regards the generation of oligodendroglial lineage cells, to date only a few protocols have resulted in the successful generation of human stem cell derived OL. Furthermore, these protocols require long culture periods (70 to 150 days) and show relatively low efficiencies⁹⁻¹².

Therefore, it would be desirable to provide a method for efficiently generating human oligodendroglial lineage cells from various cell types in large quantities.

The inventors of the present invention found a rapid and efficient protocol that facilitates the generation of human oligodendroglial lineage cells from human iPSC-derived neural progenitor cells (NPC)¹³ using the transcription factor (TF) SOX10. Using a combination of three TFs, namely SOX10, OLIG2, and NKX6.2, this can even be achieved within 28 days with an efficiency of up to 70%. Furthermore, 30% of the O4⁺ OL differentiate into mature myelin basic protein positive (MBP⁺) OL within seven additional days. The global gene expression pattern of O4⁺ OL is comparable to that of human primary OL (pOL). The induced human oligodendroglial lineage cells (iOL) are suitable for in vitro myelination assays using nanofibers or iPSC-derived neurons. After transplantation into MBP deficient shiverer mice (Shi/Shi Rag2^{-/-}) iOL disperse widely and myelinate the developmental central nervous system (CNS) as well as the adult demyelinated spinal cord. Furthermore, iOL can be used for disease modeling and to test the potential of pharmacological compounds in promoting oligodendroglial differentiation.

The invention is further defined by the embodiments reflected in the claims, described in the description, and illustrated in the Examples and Figures.

The present invention relates to a method of generating oligodendroglial lineage cells, the method comprising the steps of:
(a) providing human cells selected from the group consisting of neural progenitor cells (NPCs), pluripotent stem cells (PSCs), induced pluripotent stem cells (iPSCs) and fibroblasts;
(b) inducing and/or increasing expression of the transcription factor SOX10, optionally in combination with OLIG2 and/or NKX6.2 in the cells;
(c) culturing the cells; thereby generating the oligodendroglial lineage cells.

Furthermore, the invention relates to cells obtainable by this method, preferably wherein the cells are O4⁺ and/or MBP⁺.

In addition, the invention relates to a recombinant vector comprising a nucleotide sequence encoding SOX10, OLIG2 and NKX6.2.

The invention also relates to a human NPC, PSC, iPSC or fibroblast comprising one or more exogenous nucleic acid(s) encoding at least one or more of SOX10, OLIG2 and NKX6.2.

Furthermore, the invention relates to a method of screening for a compound promoting oligodendroglial differentiation and/or maturation, the method comprising the steps of:
(a) providing human cells selected from the group consisting of NPCs, PSCs, iPSCs and fibroblasts, optionally comprising one or more exogenous nucleic acid(s) encoding at least one or more of SOX10, OLIG2 and NKX6.2;
(b) inducing and/or increasing expression of the transcription factor SOX10, optionally in combination with OLIG2 and/or NKX6.2 in the cells;
(c) culturing the cells for a pre-determined amount of time following inducing and/or increasing expression, wherein a first sample of the cells is cultured in the presence of a compound to be tested and a second sample of the cells is cultured in the absence of the compound;
(d) determining the percentage of cells which are positive for a marker of an oligodendrocyte developmental stage in the first sample and in the second sample;
   wherein a higher percentage of cells which are positive for the marker in the first sample than in the second sample indicates that the compound promotes oligodendroglial differentiation and/or maturation.

The invention further relates to a use of the cells of the present invention in a screening method or in expression profiling or in disease modeling.

The invention further relates to a pharmaceutical composition comprising the cells of the present invention, preferably for use as a medicament.

The invention further relates to the cells of the present invention for use as a medicament.

### DESCRIPTION OF THE FIGURES

FIG. 1. Screening for oligodendroglial lineage inducing TF in human NPC

Human iPSC-derived NPC were infected with individual OL-specific TFs or RFP control virus. (a-f) OL-lineage commitment of infected iPSC-derived NPC was analysed O4 days after transgene induction by immunostaining using the OL-specific antibody O4 (green). Nuclei were counterstained with Hoechst (blue). (a) Control cultures did not express the O4 epitope. (b) SOX10 was the only tested TF inducing O4⁺ OL. (c) Addition of OLIG2 enhanced the OL-lineage commitment (d) whereas ASCL1 led to a decreased number of O4⁺ iOL. (e) Co-expression of SOX10, OLIG2 and NKX6.2 increased the number of O4⁺ cells (f) accompanied by the appearance of iOL with a more mature oligodendroglial morphology.

(g+h) Quantification of O4⁺ iOL over all cells with indicated TF combinations two weeks after transgene induction. Data are presented as mean of replicates from three independent experiments+SD. One-way ANOVA with Bonferroni's multiple comparisons test was used as statistical test (*p<0.05, **p<0.01, ***p<0.001). Scale bars: 50 μm (a-e), 25 μm (f).

FIG. 2. SOX10, OLIG2 and NKX6.2 induce a rapid and efficient oligodendroglial lineage commitment

(a) Schematic presentation of the lentiviral expression vector used for the polycistronic expression of SOX10, OLIG2 and NKX6.2. (b) Schematic summary of the differentiation protocol developed in this study using NPC expansion medium (NPCM), glial induction medium (GIM) and differentiation medium (DM).

(c-f) Representative immunofluorescence images of different NPC and OL markers during differentiation. Nuclei were counterstained with Hoechst (blue). (c) iPSC-derived NPC homogenously expressed the neural progenitor marker NESTIN (green) and SOX1 (red). (d) Seven days after transgene induction NG2⁺ as well as O4⁺ oligodendroglial lineage cells were detected. (e) By day 28, iOL expressed the O4-epitope as well as the more mature OL marker GALC and presented with a branched morphology. (f) Further maturation led to the emergence of MBP⁺ mature iOL forming myelin sheaths. (g) Representative flow cytometry analyses for the expression of O4 and RFP in control and SON cultures seven and 28 days after transgene induction. Notably, the proportion of RFP⁺ cells increased over time from 49.6 to 71.6% in SON transduced cultures suggesting that transduced cells still proliferated. (h) Quantification of O4⁺ cells in control and SON cultures one to four weeks after transgene induction. Data are presented as mean of replicates from four independent experiments each utilizing NPC derived from an independent human pluripotent stem cell line+SD.

(i) Quantification of O4⁺ iOL at day 28 derived from one human ESC and three independent iPSC lines. Data are presented as mean of replicates from three to five independent differentiation experiments per cell line+SD.

(j) Quantification of SOX1⁺ iPSC-derived NPC, (k) TUJ1⁺ neurons and (I) GFAP⁺ astrocytes in control and SON cultures 28 days after transgene induction. Data are presented as mean of replicates from three independent differentiation experiments+SD. Student's t test was performed for statistical analysis (***p<0.001).

(m) Representative immunofluorescence image of O4⁺ iOL (green) 28 days after the induction of SON either expressing (filled arrowhead) or silencing (empty arrowhead) the transgenes as identified by presence or absence of the RFP reporter respectively.

(n) Immunostaining of iOL for O4 (purple) and the proliferation marker KI67 (green) at day 14 after transgene induction. (o) Quantification of KI67⁺ transgene expressing cells (RFP) and of (p) KI67⁺/O4⁺ iOL at day 14 and 28 after induction. (p-o) Data are presented as mean of replicates from three independent differentiation experiments+SD.

Scale bars: 50 μm (c-f), 25 μm (m,n).

FIG. 3. Global transcriptional profiling of iOL

(a) Hierarchical clustering of whole genome expression profiles of iPSC (black), iPSC-derived NPC (green), iOL (red) and primary human adult OL (pOL, blue) revealed a strong correlation between iOL and pOL.

(b−c) Pairwise scatterplot analysis of log₂ adjusted global gene expression values of iPSC-derived NPC and their corresponding iOL (n=10). Genes presenting with a <2-fold difference in gene expression are illustrated in grey. (b) Characteristic OL-enriched genes were upregulated in iOL (c) whereas characteristic NPC-enriched genes were down regulated.

(d) Heatmap illustrating gene expression for cell-type enriched genes comparing iPSC-derived NPC, iOL and pOL. Each biological replicate of NPC and iOL presents the mean of two to three independent experiments.

(e-f) Venn diagram showing the overlap of genes significantly upregulated (e) or downregulated (f) in four biological independent iOL cell lines compared to their corresponding iPSC-derived NPC population. Each iOL cell line presents the mean of replicates from two to three independent experiments.

FIG. 4. iOL differentiate into mature OL and ensheath iPSC-derived neurons in vitro

(a) 35 days after transgene induction, O4⁺ iOL presented a branched morphology typical for mature OL and (b-d) expressed the mature oligodendroglial markers CNP, MAG and MBP.

(e) Quantification of mature MBP⁺ iOL over all O4⁺ iOL. Data are presented as mean of replicates from four independent differentiation experiments+SD.

(f) Immunostaining of iOL 14 days after replating on three-dimensional nanofiber scaffolds illustrating the formation of myelin sheaths around nanofibers. Nuclei are counterstained with Hoechst.

(g−h) Human in vitro myelination assay: co-culture of O4⁺ iOL purified at day 21 by MACS with iPSC-derived neurons for three weeks. (g) 3D reconstruction of confocal images for MBP (green) and the neuronal marker TUJ1 (red) suggesting wrapping of axons. Nuclei were counterstained with Hoechst (blue). (h) 3D illustration of MBP and TUJ1 colocalization (white) from the same detail.

Scale bars: 100 μm (a), 20 μm (b+c), 50 μm (d), 10 μm (f-h).

FIG. 5. iOL give rise to functional myelin 16 weeks following engraftment in brains of newborn mice

(a) Transplantation of iOL into the corpus callosum of newborn Shi/Shi Rag2^{-/-} mice resulted in extensive generation of MBP⁺ myelin (green) by human cells expressing RFP and staining positive for the human nuclei marker STEM101 (red). (b) Higher magnification of the boxed area in a. (c) Although RFP expression was downregulated in a large proportion of grafted cells following their final differentiation, confocal images revealed co-expression of MBP (ci) with RFP⁺ ensheathing cells (cii). (d) Co-labeling of MBP (blue) and neurofilaments (NF, green), suggesting wrapping of host axons (dii) by donor-derived myelin (di). (e) Axoglial elements visualized by CASPR (red), a paranodal marker, revealing functionality of donor-derived myelin (blue). Insets show staining for MBP (ei) and CASPR (eii) (arrowhead in (e)). (f) Electron microscopy images demonstrate that human-derived myelin undergoes final maturation via compaction. Axons surrounded by compact myelin are indicated by yellow stars. (fi) and (fii) are higher magnifications of boxed axon in (f). n=4 for immunostaining, n=3 for EM. Scale bars: 100 μm (a), 50 μm (b), 20 μm (c), 5 μm (d+e), 2 μm (f), 500 nm (fi) and 200 nm (fii).

FIG. 6. Functional differentiation of iOL into bona-fide mature re/myelinating OL 12 weeks following transplantation in adult demyelinated mice

(a) Coronal serial sections illustrating widespread distribution of iOL derived MBP⁺ myelin after engraftment into the dorsal funiculus (highlighted by dotted line) of the adult demyelinated Shi/Shi Rag2^{-/-} spinal cord. Grafted human cells not only remyelinated the lesion site by producing high amounts of MPB⁺ myelin (green), but also myelinated axons throughout the spinal cord white and grey matters. (b−c) Depicting cross sections of myelin sheaths (green) generated by iOL (revealed by RFP or immunopositivity for STEM101 (red)) in the (b) dorsal lesion site and (c) ventral white matter. (d) Numerous round MBP⁺ myelin-like structures co-labeled with STEM101⁺/RFP⁺. (di) and (dii) illustrate a representative RFP⁺ human oligodendrocyte (di) connected to several MBP⁺ myelin sheaths (dii). (e) Confocal image showing several MBP⁺ myelin sheaths (green) surrounding host axons (blue) in the dorsal funiculus. (f) Co-staining for human cytoplasmic/human nuclei (STEM121/STEM101 in green) revealed that many human cells were connected to MBP⁺ myelin-like structures (red). Insets in (fi)-(fiii) show individual and merged immunohistochemistry. (g−h) Longitudinal and cross views of functional human-derived myelin (green) co-labeled with neurofilaments (blue) and integrated into Node's of Ranvier revealed by paranodal marker CASPR (red) in adult spinal cord. n=4 mice for all staining. Scale bars: 200 μm (a), 20 μm (b-f), 5 μm (g+h).

FIG. 7. iOL are suitable to test the differentiation promoting effects of selected compounds and MAPT-OL exhibit mutation related phenotypes

(a and c) Representative immunofluorescence images of iOL cultures treated with either vehicle (0.01% (v/v) DMSO), thyroid hormone (T3) as a positive control or 1 μM of the drug candidate miconazole for 21 days in minimum differentiation medium (DM). Oligodendroglial lineage commitment was assessed by (a) O4 (green) and (c) MBP (green) immunostaining; nuclei were counterstained with Hoechst (blue). Quantification of (b) O4⁺ and (d) MBP⁺ iOL after treatment with either vehicle, T3 or the drug candidate dissolved in DMSO at three different concentrations (0.5 μM, 1 μM, 5 μM) for 21 days in minimum DM. Data are presented as mean of replicates from three independent experiments+SD. One-Way ANOVA with Dunnett's multiple comparisons test was performed for statistical analysis comparing the mean of each sample with DMSO control (*p<0.05, ***p<0.001). 0*=Toxic culture condition.

(e) Immunostaining for O4 (green) demonstrating differentiation of iPSC carrying the N279K MAPT mutation (MAPT1, MAPT2) and genetic corrected controls (MAPT1 GC, MAPT2 GC) into iOL. Nuclei were counterstained with Hoechst (blue). (f) Flow cytometry based quantification of O4⁺ iOL after 28 days of differentiation in MAPT mutation cultures, genetic corrected cultures and an independent healthy control culture. Data are presented as mean of replicates from three independent experiments+SD. (g) qRT-PCR analysis on control, MAPT gene corrected and MAPT mutated iOL cultures for 4R TAU isoforms containing exon 10. Expression levels were normalized to total TAU expression and control lines. Data are presented as mean of replicates from three independent experiments+SD. One-way ANOVA with post hoc Tukey test was performed for statistical analsyses (**p<0.01, ***p<0.001).

(h) Quantification of cleaved CASPASE 3⁺ iOL in Ctrl and MAPT cultures after 48 h of either vehicle (0.01% (v/v) DMSO) or rotenone treatment. Data are presented as mean of replicates from three independent experiments+SD. One-way ANOVA with post hoc Tukey test was performed for statistical analysis (*p<0.05, **p<0.01). (i) All results combined after normalization by setting all control cultures to 100%, show that MAPT N279K causes a higher sensitivity to oxidative stress. Error bars present SD. Student's t test was performed for statistical analysis (***p<0.001). Scale bars: 50 μm (a,c), 25 μm (e).

FIG. 8. Immunocytochemical analysis of pOL used for whole genome expression analysis Representative immunofluorescence image of pOL obtained from adults undergoing surgical resections as treatment for non-tumor-related intractable epilepsy after six days in vitro. The vast majority of cells were O4 (red) and MBP (green) positive. Nuclei were counterstained with Hoechst (blue). Scale bar: 50 μm.

FIG. 9. Confocal analysis of in vitro myelination assays

(a) Confocal immunofluorescence image of iOL co-cultured with iPSC-derived neurons for 21 days. The image illustrates the co-localization of MBP (green) with neuronal processes visualized by TUJ1 (red). Nuclei were counterstained with Hoechst. No MBP expression was detectable in control cultures. (b) Orthogonal projection illustrates the formation of MBP⁺ (green) sheaths around TUJ1⁺ (red) neuronal processes. Scale bars: 25 μm (a), 1 μm (b).

FIG. 10. In demyelinated spinal cord many axons are wrapped by human cells yet not remyelinated

In sections remote from the lesion center axons (blue) were found to be wrapped by RFP⁺ sheaths which were not yet MBP⁺ (green), indicating a prospective larger remyelination potential of the grafted iOL. n=4 mice, Scale bar: 20 μm.

FIG. 11. Human myelin integrates very well among endogenous myelin in adult mice

(a) Coronal serial sections from the adult Shi/Shi Rag2⁻¹ mice stained for MOG (green) and MBP (red) to reveal endogenous and exogenous myelin, respectively. (b) Higher magnification shows that MBP⁺ myelin was broadly distributed and tightly dispersed among endogenous myelin in the demyelinated adult spinal cord 12 wpg. n=4 mice, Scale bars; A: 200 μm, B: 50 μm.

FIG. 12. Schematic presentation of the polycistronic all-in-one SON lentiviral vector The human cDNAs encoding SOX10, OLIG2 and NKX6.2 were linked by 2A self-cleavage sites and were inserted into a third generation lentiviral expression vector equipped with the retroviral SFFV U3 promoter. For the visualization of transgene expression, an IRES-dTomato cassette was introduced following the SON expression cassette.

FIG. 13. SON transdifferentiates human fibtoblasts to oligodendrocytes.

Human dermal fibroblasts were either transduced with SON or, as a control, with RFP expressing lentivirus. Morphological changes were observed ten days post SON transduction whereas RFP transduced cells presented with unchanged morphologies. At day 46 of differentiation, SON transduced cells expressed the oligodendroglial marker A2B5, NG2 and O4 identified by immunocytochemistry. In contrast, control cell populations did not express any of these marker. qRT-PCR demonstrated upregulation of the OPC marker NG2 and the late OL marker MBP.

The present invention relates to a method of generating oligodendroglial lineage cells, the method comprising the steps of:
(a) providing human cells selected from the group consisting of neural progenitor cells (NPCs), pluripotent stem cells (PSCs), induced pluripotent stem cells (iPSCs) and fibroblasts;
(b) inducing and/or increasing expression of the transcription factor SOX10, optionally in combination with OLIG2 and/or NKX6.2 in the cells;
(c) culturing the cells; thereby generating the oligodendroglial lineage cells.
   Preferably, the method comprises the step of inducing and/or increasing expression of the transcription factor SOX10 in combination with OLIG2 and/or NKX6.2 in the cells, thereby further increasing the efficiency of the methods of the invention.

In a preferred embodiment, the oligodendroglial lineage cells express one or more markers selected from the group consisting of PDGFRA, ST8SIA1, NG2, O4, GALC, O1, PLP, MBP, CNP, MAG, OLIG1, MOG, and a combination thereof.

In accordance with the present invention, “SOX10” refers to the human transcription factor SOX10 represented by the NCBI reference NP_008872.1 (SEQ ID NO: 7). This protein is encoded by the SOX10 gene represented by the NCBI reference NG_007948.1. The terms SOX10 and SOX10 also comprise any fragments and variants thereof having a comparable biological activity or encoding a protein having a comparable biological activity, respectively.

In accordance with the present invention, “OLIG2” refers to the human transcription factor OLIG2 represented by the NCBI reference NP_005797.1 (SEQ ID NO: 8). This protein is encoded by the OLIG2 gene represented by the NCBI reference NG_011834.1. The terms OLIG2 and OLIG2 also comprise any fragments and variants thereof having a comparable biological activity or encoding a protein having a comparable biological activity, respectively.

In accordance with the present invention, “NKX6.2” refers to the human transcription factor NKX6.2 represented by the NCBI reference NP_796374.1 (SEQ ID NO: 9). This protein is encoded by the NKX6.2 gene represented by the NCBI reference NM_177400.2. The terms NKX6.2 and NKX6.2 also comprise any fragments and variants thereof having a comparable biological activity or encoding a protein having a comparable biological activity, respectively.

As used herein, “inducing and/or increasing expression of a transcription factor” relates to any measures suitable for increasing the amount of the corresponding transcription factor produced by the cells compared to endogenous expression.

In a preferred embodiment, the expression of one or more of the transcription factors SOX10, OLIG2 and NKX6.2 in step (b) is increased compared to endogenous expression of the corresponding transcription factors. This can be achieved by any means suitable for enhancing and/or inducing the transcription of a gene encoding the corresponding transcription factor and/or enhancing the translation of the mRNA encoding the corresponding transcription factor.

For example, endogenous transcription or translation of the transcription factor can be enhanced, e.g. by adapting the culture conditions to favor expression of the transcription factor and/or by contacting the cell with a compound capable of such enhancing. It is also possible to genetically modify the cell in order to induce and/or increase production of the corresponding transcription factor, e.g. by introducing a nucleic acid encoding the corresponding transcription factor. In general, any measures useful in achieving the goal of increasing the amount of the corresponding transcription factor produced by the cells compared to endogenous expression can be used according to the present invention.

In a preferred embodiment, the expression of one or more of the transcription factors SOX10, OLIG2 and/or NKX6.2 is an ectopic expression. The term ectopic expression refers to a situation wherein a cell expresses a protein which it normally would not express in a given situation. For example, such lack of expression could be due to physiological downregulation of the corresponding gene. In order to initiate ectopic expression, a cell can be genetically manipulated, e.g. by introducing an alternative promoter such as a constitutive or inducible promoter or by introducing a nucleic acid comprising a nucleotide sequence encoding the corresponding gene product and optionally a corresponding promoter enabling increased expression compared to endogenous expression.

In a preferred embodiment, one or more nucleic acid(s) comprising one or more nucleotide sequence(s) encoding one or more of the transcription factors SOX10, OLIG2 and NKX6.2 is/are introduced in the cells of step (a). Such nucleic acid which originates outside of the cell in which it is introduced is also termed an exogenous nucleic acid. For example, the exogenous nucleic acid can integrate in the genome of the cell or it can remain a distinct entity within the cell. In addition to genes encoding the corresponding transcription factors, the nucleic acid(s) can comprise further transcriptional regulatory elements such as one or more promoters suitable for mediating expression of the transcription factors. It is possible to use inducible and/or constitutive promoters. Constitutive promoters are largely independent of environmental and/or developmental factors and generally provide for stable expression of the genes they control. The activity of inducible promoters is dependent on environmental conditions and external stimuli. They enable controllability of expression of the genes they control in a temporal and/or spatial manner. For example, expression can be turned on or off at a given time point by adding an external stimulus to the culturing medium. A preferred promoter suitable for use in the current invention is inducible by tetracycline.

In general, the inducible promoter can be induced for any suitable amount of time such as for at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more days, or for about 7-42 days, 10-32 days, 14-28 days or for the whole duration of time of culturing in step (c). The duration of induction of the promoter can be optimized for any given experimental setting. For example, when using NPCs, the promoter could be induced for at least 5, 6, 7, 8, 9 or 10 days, preferably for about 6-10 days, more preferably for about 7 days. For example, when using iPSCs, the promoter could be induced for about 10-14 days. For example, when using fibroblasts, the promoter could be induced for about 28-42 days.

The one or more nucleic acid(s) to be introduced in a cell can be provided on any vector suitable for gene delivery. Suitable recombinant vectors are known to the skilled person. For vector modification techniques, see Sambrook and Russel “Molecular Cloning, A Laboratory Manual”, Cold Spring Harbor Laboratory, N. Y. (2001). In general, the one or more nucleic acid(s) comprising one or more nucleotide sequence(s) encoding one or more of SOX10, OLIG2 and NKX6.2 can be present on a vector such as a non-viral vector or a viral vector. In one embodiment, the recombinant vector comprises a nucleotide sequence encoding SOX10, OLIG2 and NKX6.2. In a preferred embodiment, the vector encoding SOX10, OLIG2 and NKX6.2 is a polycistronic vector. In case of viral vectors, retroviral vectors are preferred and lentiviral vectors are especially preferred in the methods of the invention.

Introduction of the nucleic acid(s) in the cell can be conducted by any known method for gene delivery applicable for introducing nucleic acids in human cells. For example, non-viral or viral methods are suitable in the present invention. Non-viral gene delivery methods comprise electroporation, microinjection, gene gun, impalefection, hydrostatic pressure, continuous infusion, protein transduction and sonication and chemical methods such as lipofection. In a preferred embodiment of the present invention viral methods for introducing nucleic acids in human cells are used. According to this embodiment, the one or more nucleic acid(s) encoding one or more of the transcription factors SOX10, OLIG2 and NKX6.2 and optionally one or more corresponding promoters are present on a recombinant viral vector which is suitable for transduction of human cells.

As used herein, the term “oligodendroglial lineage cells” refers to a type of glial cells and comprises oligodendrocytes, also referred to as oligodendroglia, of any developmental stage. As such, this term comprises oligodendrocyte precursor cells (OPCs), differentiated oligodendrocytes, mature oligodendrocytes and myelinating oligodendrocytes. Markers which can be used to identify or differentiate these cells are generally known to a skilled person⁵². Exemplary markers for various oligodendroglial lineage cells are PDGFRA, ST8SIA1, NG2, O4, GALC, O1, PLP, MBP, CNP, MAG, OLIG1 and MOG.

In the methods of the present invention, different human cell types can be used for the generation of oligodendroglial lineage cells. Useful cell types comprise neural progenitor cells (NPCs), pluripotent stem cells (PSCs), induced pluripotent stem cells (iPSCs) and fibroblasts, while NPCs and iPSCs are preferred, and NPCs derived from PSCs or iPSCs are especially preferred.

Preferably, the pluripotent stem cells are induced pluripotent stem cells (iPSCs) which can be generated by any method known in the art. In general, iPSCs may be obtained from any adult somatic cell (of a subject). Exemplary somatic cells include peripheral blood mononuclear cells (PBMCs) from blood or fibroblasts such as fibroblasts obtained from skin tissue biopsies. For example, iPSCs can be generated as described by Reinhardt et al.¹³ or Ehrlich et al.¹⁷, which disclosures are hereby incorporated by reference.

NPCs can be generated by any method known in the art. For example, NPCs can be derived from iPSCs by treatment with small molecules as described in the Examples accompanying the description and by Reinhardt et al.¹³ or Ehrlich et al.¹⁷, which disclosures are hereby incorporated by reference.

In general, the origin of the cells used in the methods of the present invention is generally not decisive, i.e. it is possible to use cells of any origin, e.g., native or primary cells or cell lines. However, in certain embodiments the use of native or primary cells or the use of cells derived therefrom is preferred. This approach enables the generation of patient-specific oligodendroglial lineage cells in the methods of the present invention and is especially useful when preparing oligodendroglial lineage cells for use in therapy. Furthermore, cells useful in the methods of the present invention can be cells which are freshly prepared or can be cells which have been stored under suitable conditions. For example, human iPSC-derived NPC can be frozen and cost-efficiently expanded¹⁷.

The method of generating oligodendroglial lineage cells of the present invention can be used to generate a variety of cell types reflecting various developmental stages of oligodendroglial lineage cells. Markers useful in the methods of the invention include PDGFRA, ST8SIA1, NG2, O4, GALC, O1, PLP, MBP, CNP, MAG, OLIG1 and MOG, but the invention is not limited to these specific markers.

PDGFRA (NP_006197.1) is a marker for OPC.

ST8SIA1 (NP_001291379.1, NP_003025.1) is a marker for OPC.

NG2 (NP_001888.2; Gene name: CSPG4) is a marker for OPC.

O4 (epitope not assigned to any protein; Sommer et al., Dev Biol. 1981 Apr. 30; 83(2):311-27) is a marker for late OPC, early OL.

O1 (NP_000144.2, NP_001188330.1, NP_001188331.1; epitope is assigned to GALC) is a marker for OL.

GALC (NP_000144.2, NP_001188330.1, NP_001188331.1) is a marker for OL.

PLP (NP_000524.3, NP_001122306.1, NP_001291933.1, NP_955772.1) is a marker for OL.

MBP (NP_001020252.1, NP_001020261.1, NP_001020263.1, NP_002376.1) is a marker for mature OL.

CNP (NP_149124.3) is a marker for OL.

MAG (NP_001186145.1, NP_002352.1, NP_542167.1) is a marker for mature OL.

OLIG1 (NP_620450.2) is a marker for OL.

MOG (9 isoforms; NP_001008229.1) is a marker for mature OL.

In a preferred embodiment, the oligodendroglial lineage cells generated in the method of the present invention express at least one marker selected from the group consisting of PDGFRA, ST8SIA1, NG2, O4, GALC, O1, PLP, MBP, CNP, MAG, OLIG1, MOG, and a combination thereof. In an especially preferred embodiment, the oligodendroglial lineage cells generated in the method of the present invention express O4, optionally in combination with one or more markers selected from the group consisting of PDGFRA, ST8SIA1, NG2, GALC, O1, PLP, MBP, CNP, MAG, OLIG1 and MOG.

In a preferred embodiment, the oligodendroglial lineage cells generated in the method of the present invention belong to one or more developmental stages selected from oligodendrocyte precursor cells (OPCs), differentiated oligodendrocytes, mature oligodendrocytes, myelinating oligodendrocytes and combinations thereof.

In various embodiments, the oligodendroglial lineage cells generated in the method of the present invention express one or more markers selected from the group consisting of PDGFRA, ST8SIA1, NG2 and O4. In various embodiments, the oligodendroglial lineage cells generated in the method of the present invention express one or more markers selected from the group consisting of O4, O1, GALC, PLP, CNP and OLIG1. In various embodiments, the oligodendroglial lineage cells generated in the method of the present invention express one or more markers selected from the group consisting of MBP, MAG and MOG.

The skilled person is aware of suitable methods of determining whether one or more of the above recited markers are expressed by the cells generated by the methods of the present invention. Exemplary methods are also described in the Examples herein below. Such methods for detecting markers include, without being limiting, determining the expression of a marker on the amino acid (polypeptide) level as well as on the nucleic acid molecule level. The present invention also envisions that nucleic acid molecules encoding proteins as described herein, as well as RNA and proteins as described herein can be detected by e.g. RNA and protein analysis, e.g. by immunocytochemical analysis.

The term “nucleic acid” or “nucleic acid molecule”, when used herein, encompasses any nucleic acid molecule having a nucleotide sequence of bases comprising purine- and pyrimidine bases, wherein said bases represent the primary structure of a nucleic acid molecule. Nucleic acid sequences can include DNA, cDNA, genomic DNA, RNA, both sense and antisense strands. The polynucleotide of the present invention can be composed of any polyribonucleotide or polydeoxyribonucleotide, which may be unmodified RNA or DNA or modified RNA or DNA.

Methods for the determining of expression levels of a marker on the amino acid level include but are not limited to immunohistochemical methods as described in the appended examples but also other methods, e.g. western blotting or polyacrylamide gel electrophoresis in conjunction with protein staining techniques such as Coomassie Brilliant blue or silver-staining. Also of use in protein quantification is the Agilent Bioanalyzer technique. Further methods of determination of expression levels of a marker include, without being limiting, cell sorting approaches such as magnetic activated cell sorting (MACS) or flow cytometry activated cell sorting (FACS) or panning approaches using immobilised antibodies as described for example in Dainiak et al. (Adv Biochem Eng Biotechnol. 2007; 106:1-18⁵³). Methods for determining the expression of a protein on the nucleic acid level include, but are not limited to, northern blotting, PCR, RT-PCR or real time PCR as well as techniques employing microarrays. All these methods are well known in the art and have been described in part in the appended examples.

All of the definitions and procedures provided hereinabove in the context of markers which are expressed by the cells of the invention apply mutatis mutandis to markers that are downregulated or not expressed in the cells of the invention.

It is further envisioned by the present invention that also variants and fragments of the markers as described herein can be detected.

As used herein, a “variant of a polypeptide” encompasses polypeptides having amino acid sequences which differ in one or more amino acids from the amino acid sequence of the polypeptide from which they are derived. These differences can be due to, e.g., deletions, insertions, inversions, repeats, and substitutions of one or more amino acids. Variants have a comparable biological activity to the polypeptides from which they are derived, i.e. they have essentially the same functional properties.

A “variant of a nucleic acid molecule” of the present invention encompasses nucleic acids having nucleotide sequences which differ in one or more nucleotides from the nucleotide sequences of the nucleic acid from which they are derived. These differences can be due to deletions, insertions and substitutions of one or more nucleotides. In general, such nucleic acid variants have a sequence encoding polypeptides falling within the above definition of polypeptide variants, i.e. which have a comparable biological activity to the polypeptides from which they are derived.

Similarly, a “fragment” as used herein can be any nucleic acid molecule or polypeptide which comprises a deletion of 1, 2, 3, 4, 5, 10, 20, 30 or more amino acid residues of the polypeptide from which the fragment is derived or a deletion of more than 1, 2, 3, 4, 10, 20, 30, 40, 50, 60, 70, 80, 90, 100, 200, 300 or more nucleotides compared to a nucleic acid from which the fragment is derived. The fragment may still have the same functional properties as any of the polypeptides or the nucleic acid molecules from which the fragment is derived. Given that also variants and fragments of the markers (polypeptides, nucleic acid molecules) as described herein are encompassed by the present invention, the present invention also encompasses detection of sequences which have a sequence identity of 80%, 85%, 90%, 95%, 97%, 99% or 100% with any of the polypeptides/nucleic acid molecules described hereinbefore.

In accordance with the present invention, the term “identical” or “percent identity” in the context of two or more nucleic acid molecules or amino acid sequences, refers to two or more sequences or subsequences that are the same, or that have a specified percentage of amino acid residues or nucleotides that are the same (e.g., at least 95%, 96%, 97%, 98% or 99% identity), when compared and aligned for maximum correspondence over a window of comparison, or over a designated region as measured using a sequence comparison algorithm as known in the art, or by manual alignment and visual inspection. Sequences having, for example, 80% to 95% or greater sequence identity are considered to be substantially identical. Such a definition also applies to the complement of a test sequence. Those having skill in the art will know how to determine percent identity between/among sequences using, for example, algorithms such as those based on the CLUSTALW computer program (Thompson Nucl. Acids Res. 2 (1994), 4673-4680⁵⁴) or FASTDB (Brutlag Comp. App. Biosci. 6 (1990), 237-245⁵⁵), as known in the art.

For example, BLAST2.0, which stands for Basic Local Alignment Search Tool (Altschul, Nucl. Acids Res. 25 (1997), 3389-3402⁵⁶; Altschul, J. Mol. Evol. 36 (1993), 290-300⁵⁷; Altschul, J. Mol. Biol. 215 (1990), 403-410⁵⁸), can be used to search for local sequence alignments.

Oligodendroglial lineage cells generated according to the methods of the present invention can comprise more than one population of cells. Certain percentages of the generated cells can express specific markers, i.e. the cells can be positive for said markers. Depending on the requirements of the application the generated cells are intended for, the generated oligodendroglial lineage cells can optionally be further purified or isolated, i.e. populations of cells differing in marker expression can be separated. Identification and optional purification of oligodendroglial lineage cells expressing a given marker can be carried out by any suitable method in the art, e.g. by methods employing antibodies which specifically bind to these markers. Potentially useful markers comprise PDGFRA, ST8SIA1, NG2, O4, GALC, O1, PLP, MBP, CNP, MAG, OLIG1 and MOG.

In various embodiments of the present invention the cells are cultured in step (c) for a pre-determined amount of time following inducing and/or increasing expression of the transcription factor(s) in order to generate the oligodendroglial lineage cells. The time point of “inducing and/or increasing expression” can be defined as the time point at which expression of the corresponding transcription factor(s) is increased compared to endogenous expression of the transcription factor. The duration of time of culturing in step (c) can be adapted individually, for example according to the desired marker expression of the oligodendroglial lineage cells, the desired percentage of cells expressing a specific marker, or any other relevant circumstances. For example, the cells can be cultured in step (c) for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 days or more. The duration for which the cells are cultured following inducing and/or increasing expression has an impact on the differentiation status of the oligodendroglial lineage cells, i.e. on expression of specific markers such as O4 and/or MBP.

In a preferred embodiment, after culturing the cells in step (c) for 7 days following inducing and/or increasing expression, at least 5%, preferably at least 6%, more preferably at least 7%, still more preferably at least 8% of the cells are O4⁺ oligodendroglial lineage cells.

In a preferred embodiment, after culturing the cells in step (c) for 14 days following inducing and/or increasing expression, at least 15%, preferably at least 16%, more preferably at least 17%, still more preferably at least 18% of the cells are O4⁺ oligodendroglial lineage cells.

In a preferred embodiment, after culturing the cells in step (c) for 21 days following inducing and/or increasing expression, at least 30%, 31% or 32% preferably at least 33%, 34% or 35%, more preferably at least 36%, 37% or 38%, still more preferably at least 39%, 40% or 41% of the cells are O4⁺ oligodendroglial lineage cells.

In a preferred embodiment, after culturing the cells in step (c) for 28 days following inducing and/or increasing expression, at least 55%, 56%, 57% or 58%, preferably at least 59%, 60%, 61% or 62%, more preferably at least 63%, 64%, 65% or 66%, still more preferably at least 67%, 68% or 69% of the cells are O4⁺ oligodendroglial lineage cells.

In a preferred embodiment, after culturing the cells in step (c) for about 35 days following inducing and/or increasing expression, at least 20%, 21%, 22%, 23% or 24%, preferably at least 25%, 26%, 27%, 28% or 29%, more preferably at least 30%, 31%, 32%, 33% or 34%, still more preferably at least 35%, 36%, 37% or 38% of O4⁺ oligodendroglial lineage cells are also MBP⁺.

In a further preferred embodiment MBP⁺ oligodendroglial lineage cells generated according to the methods of the invention comprise a subpopulation of cells which are also positive for the mature oligodendroglial markers CNP and MAG.

Additionally, the oligodendroglial lineage cells generated according to the methods of the present invention have a different global gene expression profile compared to the cells provided in step (a). For example, expression of GALC, OLIG1, MOG and/or MBP can be upregulated in the generated oligodendroglial lineage cells compared to the cells provided in step (a), such as NPCs, and/or expression of SOX1, PAX6 and/or PAX7 can be downregulated in the generated oligodendroglial lineage cells compared to the cells provided in step (a), such as NPCs. In certain embodiments the oligodendroglial lineage cells can also express PDGFRA and/or ST8SIA1. In certain embodiments this altered gene expression profile can be observed about 14, 16, 28, 20, 22, 24, 26, 28, 30, 32 or 34 days following inducing and/or increasing expression, preferably about 22-32 days, more preferably about 28 days following inducing and/or increasing expression, at the latest.

The methods of the present invention further require that cells such as iPSCs, PSCs, NPCs or fibroblasts are cultivated. In general, the methods of the present invention can be carried out in any cell culture. Culture conditions may vary, but the artificial environment in which the cells are cultured often comprise a suitable vessel comprising one or more of the following: a substrate or medium that supplies the essential nutrients (amino acids, carbohydrates, vitamins, minerals), growth factors, hormones, gases (O₂, CO₂) and/or regulated physico-chemical environment (pH, osmotic pressure, temperature). Cell culture as described herein refers to the maintenance and growth of cells in a controlled laboratory environment. Such in vitro cell culture models are well-known in experimental cell biological research. For example, cells can be cultured while attached to a solid or semi-solid substrate (adherent or monolayer culture). Cells can also be grown floating in the culture medium (suspension culture).

In order to further promote oligodendroglial lineage differentiation in the cells provided in step (a), a medium used at least in part of step (c) can comprise an inducer of oligodendroglial lineage differentiation. However, addition of such an inducer is not essential. Exemplary inducers of oligodendroglial lineage differentiation include a thyroid hormone such as triiodothyronine (T3), miconazole or benztropine. Preferably T3 is used. Typical concentrations for T3 are in the range of about 1-100 ng/ml, 5-60 ng/ml, 10-30 ng/ml or 20-25 ng/ml.

In a preferred embodiment of the present invention, the cells are cultured in step (c) in a first medium for about 1-6 days such as about 2, 3, 4, 5 or 6 days, preferably about 2-4 days, and thereafter in a second medium. The first and the second medium can differ in the nature and/or concentration of one or more of their constituents.

In a preferred embodiment of the present invention, the second medium comprises a higher concentration of an inducer of oligodendroglial lineage differentiation than the first medium. In a more preferred embodiment, the first medium comprises about 1-30 ng/ml T3 or about 5, 10, 15, 20 or 25 ng/ml T3 and the second medium comprises about 10-100 ng/ml T3 or about 20, 30, 40, 50, 60, 70, 80 or 90 ng/ml T3. In an especially preferred embodiment the first medium comprises 5-20 ng/ml or about 10 ng/ml T3, and the second medium comprises 45-75 ng/ml or about 60 ng/ml T3.

In general, any medium capable of promoting cell growth in the methods of the present invention can be used. Exemplary media are DMEM-F12 or neurobasal medium. Preferably the medium comprises about 0.1-10 mM glutamine and optionally about 0.1-10% serum. As used herein, “serum” can comprise any suitable serum such as fetal calf serum (FCS) or fetal bovine serum (FBS). A preferred medium is DMEM-F12, optionally with N2 supplement or B27 supplement lacking vitamin A. Optionally, the medium can comprise one or more additional compounds selected from the group consisting of penicillin/streptomycin/glutamine, Smoothened agonist (SAG), Platelet-Derived Growth Factor (PDGF), Neurotrophin-3 (NT3), Insulin-like Growth Factor-I (IGF-I), ascorbic acid (AA), Trace Elements B, progesterone, putrescine, selenite, transferrin, insulin and/or activators of protein kinase A such as dbcAMP.

In a preferred embodiment the first medium and the second medium comprise DMEM-F12 comprising 0.1-10 mM glutamine and optionally 0.1-10% serum, and the second medium comprises T3.

A preferred first medium further comprises one or more of N2 supplement, B27 supplement lacking vitamin A, penicillin/streptomycin, Smoothened agonist (SAG), Platelet-Derived Growth Factor (PDGF), Neurotrophin-3 (NT3), Insulin-like Growth Factor-I (IGF-I), ascorbic acid (AA), Trace Elements B, an inducer of oligodendroglial lineage differentiation, preferably T3, progesterone, putrescine, selenite, transferrin and/or insulin.

A preferred second medium further comprises one or more of N2 supplement, B27 supplement lacking vitamin A, penicillin/streptomycin, 1-100 ng/ml T3, NT3, IGF-I, AA, Trace Elements B and activators of protein kinase A such as dbcAMP.

Highly preferred media compositions which can be used in the methods of the present inventions are detailed below, e.g., in example 1.

Oligodendroglial lineage cells generated according to the methods of the present invention can have certain phenotypic characteristics comparable to corresponding primary oligodendroglial lineage cells having similar marker expression such as oligodendrocyte precursor cells (OPCs), differentiated oligodendrocytes, mature oligodendrocytes and/or myelinating oligodendrocytes. For example, the cells generated according to the methods of the present invention can have a similar morphology or comparable myelinogenic capability as their primary counterparts. These characteristics can be analyzed in any known in vitro and/or in vivo assay and can be compared to the characteristics of corresponding primary oligodendroglial lineage cells. Exemplary in vitro and/or in vivo assays are given in examples 5 and 6 below.

Thus, in a preferred embodiment, the generated oligodendroglial lineage cells are capable of producing myelin-like sheaths surrounding axons of co-cultured iPSC-derived neurons in an in vitro assay. For example, the cells may be cultivated for about 21 days in step (c) prior to co-culturing. In another preferred embodiment, the generated oligodendroglial lineage cells are capable of remyelinating demyelinated axons in a Shi/Shi Rag2^{-/-} mouse model. For example, the cells may be cultivated for about 14 days in step (c) prior to grafting the cells in the mouse central nervous system. These assays are explained in more detail in examples 5 and 6 below.

The present invention also relates to oligodendroglial lineage cells obtainable by the methods of the present invention. These cells can be characterized as recited in the detailed description pertaining to the methods of the invention. In especially preferred embodiments these cells are O4⁺ and/or MBP⁺.

Involvement of oligodendroglial lineage cell depletion and/or damage has been shown in various neurodegenerative and/or myelin diseases. Demyelinating disorders like multiple sclerosis (MS) affect many individuals worldwide. Thus, research on neurodegenerative and/or myelin diseases represents a highly active field of research. Several approaches to counteract the negative effects caused by demyelination in patients are being studied. Among those approaches are pharmacological efforts to act directly on oligodendroglial lineage cells on the one hand and cell replacement therapies on the other hand.

As regards the pharmacological efforts, there are many conceivable ways how pharmacologically active compounds can positively influence oligodendroglial lineage cells. For example, such compounds could promote oligodendroglial differentiation and/or maturation, have protective effects on these cell types, or enhance their myelinating capabilities.

Previous efforts in this field are hampered by the lack of availability of human oligodendroglial lineage cells. While specific in vitro models and/or in vivo animal models are available to reproduce certain molecular, cellular and/or physiological aspects associated with oligodendroglial lineage cells, it must be taken into consideration that none of these models is a true reproduction of human oligodendroglial lineage cells. Especially regarding animal studies based on rodents such as mice, the lack of translatability of results obtained from these models must not be underestimated⁴⁷. Thus, while several compounds have been identified to have positive effects on oligodendroglial differentiation and/or maturation in certain in vitro models and/or in vivo animal models, the clinical value of such findings is limited.

The methods and cells of the present invention are useful in overcoming these obstacles and will provide highly useful tools for advancing these and other pharmacological efforts. For example, the methods and cells of the present invention provide a tool box for preclinical studies on human and/or even patient-specific oligodendroglial lineage cells.

In this regard, the present invention also relates to a method of screening for a compound promoting oligodendroglial differentiation and/or maturation, the method comprising the steps of:
(a) providing human cells selected from the group consisting of NPCs, PSCs, iPSCs and fibroblasts, the cells optionally comprising one or more exogenous nucleic acid(s) encoding at least one of SOX10, OLIG2 and NKX6.2;
(b) inducing and/or increasing expression of the transcription factor SOX10, optionally in combination with OLIG2 and/or NKX6.2 in the cells;
(c) culturing the cells for a pre-determined amount of time following inducing and/or increasing expression, wherein a first sample of the cells is cultured in the presence of a compound to be tested and a second sample of the cells is cultured in the absence of the compound;
(d) determining the percentage of cells which are positive for a marker of an oligodendrocyte developmental stage in the first sample and in the second sample;
   wherein a higher percentage of cells which are positive for the marker in the first sample than in the second sample indicates that the compound promotes oligodendroglial differentiation and/or maturation.

This method of screening has many method steps and features in common with the method of generating oligodendroglial lineage cells detailed above. Therefore, in addition to the following statements, any definitions and detailed explanations regarding the method of generating oligodendroglial lineage cells may also apply to the method of screening. Particularly, it should be noted that, in specific embodiments, it may be necessary to introduce one or more nucleic acid(s) comprising one or more nucleotide sequence(s) encoding one or more of the transcription factors SOX10, OLIG2 and NKX6.2 in the cells of step (a).

The compound to be tested in the method of screening is not limited to a specific class of compounds and can be any compound such as a small molecule or a polypeptide/protein. In general, any library of compounds can be screened according to the invention. In a specific embodiment, any given library can be subjected to a preselection according to specific criteria. For example, compounds known to have positive effects on oligodendroglial differentiation and/or maturation in certain in vitro models and/or in vivo animal models can be screened. Such an approach can be used to verify whether a candidate agent promotes oligodendroglial differentiation and/or maturation in human cells.

As used herein, a “candidate agent” is a compound for which there is a certain probability that is has relevant effects on oligodendroglial differentiation and/or maturation. This probability can be based on findings from in vitro models and/or in vivo animal models or it can be based on predictions resulting from literature data mining or any other studies such as structure prediction.

In a preferred embodiment, the compound to be tested is a candidate agent for treating neurodegenerative and/or myelin diseases. The term “neurodegenerative diseases” comprises a group of hereditary and sporadic conditions characterized by progressive dysfunction, degeneration and death of specific populations of neurons, which are often synaptically interconnected. The term “myelin diseases” or “demyelinating diseases” comprises a group of diseases which are associated with damage to myelin sheaths of neurons. Exemplary neurodegenerative diseases and myelin diseases include, but are not limited to, Parkinson's disease, cerebral palsy, multiple system atrophy, amyotrophic lateral sclerosis, frontotemporal dementia with Parkinsonism linked to chromosome 17 (FTDP-17), periventricular leukomalacia, Alzheimer's disease, dementia with Lewy bodies, multiple sclerosis, inflammatory demyelinating diseases and various leukodystrophies.

In general, the marker which is detected in the method of screening can be any marker of any oligodendrocyte developmental stage. In a preferred embodiment the marker is a marker for one or more of oligodendrocyte precursor cells (OPCs), differentiated oligodendrocytes, mature oligodendrocytes and/or myelinating oligodendrocytes. In a more preferred embodiment, the marker is selected from the group consisting of PDGFRA, ST8SIA1, NG2, O4, GALC, O1, PLP, MBP, CNP, MAG, OLIG1, MOG, and a combination thereof. As such, the presence of a marker in/on a cell analyzed in the method of the present invention indicates that this cell has differentiated and/or matured into a specific oligodendroglial lineage cell which can be characterized by expression of said marker.

An important advantage of the present invention is the high efficiency of providing oligodendroglial lineage cells as well as the short period of time needed for differentiation and/or maturation of the cells. These effects are also relevant for the method of screening according to the invention. Contrary to previous protocols, the amount of time needed for steps (a), (b) and/or (c), especially for step (c), is significantly reduced. On the one hand, this translates to a significant cost reduction, and on the other hand it is a crucial prerequisite for providing a method of screening in a high throughput format. Thus, in a preferred embodiment, the method of screening is a high throughput screening.

The method of screening can be adapted to various applications. For example, a given compound can be tested in various concentrations by providing more than one sample, e.g., 2, 3, 4, 5, 6, 7, 8, 9, 10 or more samples in step (c), each sample corresponding to a specific concentration of the compound. Especially in combination with the possibility of performing the screening as a high throughput screening, the method of screening is suitable for accounting for concentration-dependent effects of a compound.

Before determining the percentage of cells which are positive for a marker of an oligodendrocyte developmental stage, the cells are cultured for a pre-determined amount of time following inducing and/or increasing expression. The pre-determined amount of time can be adapted to the specific circumstances of a given assay. For example, it can be from about 5-40 days, from about 15-30 days or from about 20-25 days, or it can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 days. In an especially preferred embodiment the cells are cultured for about 15-25 days, preferably about 21 days.

The method of screening of the invention also includes one or more controls. For example, in addition to a first sample including the compound to be tested, a second sample can be included in the screening method as a negative control. Additionally, a positive control can be included, e.g., as a third sample. The positive control can include any known inducer of oligodendroglial lineage differentiation as recited hereinbefore.

In step (d), the percentage of cells which are positive for a specific marker can be determined by any known method.

As regards a second approach to counteract the negative effects caused by demyelination in a patient, namely cell replacement therapies as mentioned above, the present invention can also be used in a method of treatment, preferably in a method of treatment of a neurodegenerative and/or myelin disease.

Thus, the present invention also relates to a method of treating the diseases recited above or recited herein in a subject, comprising administering a therapeutically effective amount of a cell generated according to a method of the present invention to said subject. The “therapeutically effective amount” for each cell type can vary with factors including but not limited to the activity of the cells used, stability of the cells in the patient's body, the severity of the conditions to be alleviated, the age and sensitivity of the patient to be treated, adverse events, and the like, as will be apparent to a skilled person. The amount of cells to be administered can be adjusted as the various factors change over time. Such adjustment is well within the skill of the person skilled in the art.

In general, any cell described herein can be used as a medicament, e.g. by administering the cell to a subject suffering from a disease as recited above or recited herein and in need of ameliorating or improving symptoms. Thereby, the number of oligodendroglial lineage cells present in a subject can be increased. The subject may suffer from a neurodegenerative and/or myelin disease. As regards such cell replacement therapy, it is highly preferable to provide a subject with autologous cells, i.e. oligodendroglial lineage cells which are derived from cells obtained from the subject according to a method of the present invention.

The present invention further relates to a pharmaceutical composition comprising cells of the present invention, i.e. cells obtainable by the methods of the present invention and/or cells as described below or as described herein.

The invention further relates to the pharmaceutical composition of the present invention and/or the cells of the present invention for use as a medicament, preferably for use in the treatment of neurodegenerative and/or myelin diseases, more preferably for use in the treatment of any of the neurodegenerative and/or myelin diseases specifically mentioned above or mentioned herein.

Alternatively or additionally to the preceding explanations, the methods of the present invention, i.e. both the methods of generating oligodendroglial lineage cells, the methods of screening for a compound promoting oligodendroglial differentiation and/or maturation, and the methods of treatment, can also be carried out by providing human cells selected from the group consisting of neural progenitor cells (NPCs), pluripotent stem cells (PSCs), induced pluripotent stem cells (iPSCs) and fibroblasts in step (a), wherein the cells already comprise one or more exogenous nucleic acid(s) encoding one or more of SOX10, OLIG2 and NKX6.2.

Thus, the present invention also provides a human NPC, PSC, iPSC or fibroblast comprising one or more exogenous nucleic acid(s) encoding at least one or more of SOX10, OLIG2 and NKX6.2. In a preferred embodiment the human NPC, PSC, iPSC or fibroblast comprises one or more exogenous nucleic acid(s) encoding SOX10 and optionally OLIG2. In an especially preferred embodiment the human NPC, PSC, iPSC or fibroblast comprises one or more exogenous nucleic acid(s) encoding SOX10, OLIG2 and NKX6.2. It should be noted that the term “cell comprising an exogenous nucleic acid” also comprises cells or cell lines derived from the cell in which the exogenous nucleic acid originally has been introduced. Regarding further specifications of these cells, it is referred to the detailed description above, e.g. the description of the methods of the present invention. For example, expression of SOX10, OLIG2 and/or NKX6.2 can be under the control of one or more promoters. As such, constitutive or inducible promoters can be used, while inducible promoters are preferred. Especially preferred are promoters inducible by tetracycline.

It is also contemplated by the present invention that the cell generated according to the methods of the present invention can be used in screening, expression profiling or disease modeling.

Previously established protocols using in vitro patterning to derive OL from human iPSC or embryonic stem cells (ESCs) are characterized by long culture periods (over 120 days), relatively low efficiencies and variable reproducibility. Although these protocols have been further optimized to reduce culture times and increase efficiency, they still require at least 75 days of culture and only a small percentage of cells become MBP⁺ mature OL^{9, 10}.

Since oligodendroglial differentiation is orchestrated by a combination of individual TFs, the present inventors initially tested individual and combinations of TFs previously shown to be involved in oligodendroglial differentiation in rodents^{15, 27-31}. The results presented in the Examples section have defined that SOX10, and in particular a combination of three different TFs efficiently induces iOL and indeed overcomes the rate-limiting step of oligodendroglial specification. The inventors found that SOX10 was the only TF that induced expression of O4 in iPSC-derived NPC demonstrating that SOX10 is one key TF to induce oligodendroglial lineage commitment. Combination of SOX10 with OLIG2 and NKX6.2 further enhanced the commitment into the oligodendroglial lineage resulting in a significantly higher percentage of O4⁺ cells 14 days after induction.

To assess the reproducibility of this protocol the inventors derived iOL from three different iPSC-derived NPC lines and a single ESC-derived NPC line. The protocol was highly efficient and reproducible resulting in 50 to 70% O4⁺ cells after 28 days in all cell lines tested. Furthermore, the inventors validated the molecular profile of the derived cells with that of primary oligodendrocytes derived from surgically resected samples of adult human brain.

The myelinating capacity of iOL was tested in vitro and in vivo. In vitro iOL ensheath the neuronal process of iPSC-derived neurons as well as nanofibers confirming that physical properties of axons are sufficient to induce wrapping of axons as it has been described for rodent OL^{32, 33}. The co-culture of iOL with nanofibers facilitates the identification of compounds that exclusively promote axon ensheathment without potentially modulating molecular axonal signaling. In contrast to the in vivo transplantation experiments, the inventors did not detect CASPR accumulations indicative for paranode formation in iPSC-derived neuron/iOL co-cultures suggesting that distinct axonal signaling cascades required for the formation of paranodes and nodes are not activated in iPSC-derived neurons. In contrast to previous publications, in which fetal neural progenitors or CD140⁺ OPC were transplanted, cells were sorted prior to transplantation into Shi/Shi Rag2^{-/-} mice using the late-OPC marker O4^{11, 34, 35}. Despite this relatively mature phenotype transplanted cells not only myelinated efficiently the forebrain in newborn Shi/Shi Rag2^{-/-} mice but also remyelinated the adult demyelinated spinal cord. In adult spinal cord remyelination was not limited to the demyelinated lesions in the dorsal funiculus; the cells also dispersed extensively through the spinal cord and numerous myelinated axons were found in grey matter and ventral spinal cord. These results are similar to the reports of transplanted human fetal neural progenitors³⁶ suggesting that O4⁺ iOL are as migratory as human fetal NPC in Shi/Shi Rag2^{-/-} mice. Although the extensive remyelination potential of murine iPSC-derived NPC in the adult CNS, a condition associated with impoverished tissue plasticity and trophic support, has been recently reported³⁷, the remyelination potential of human iPSC-derived oligodendroglial lineage cells has not been demonstrated so far.

iPSC technology is an emerging tool for drug development. Promotion of remyelination represents until now an unmet treatment strategy for patients with MS. In large compound screens using rodent primary or iPSC-derived OL a number of FDA approved drugs has been identified that were able to promote oligodendroglial differentiation in vitro and remyelination in vivo^{1, 19, 20}. To determine whether iOL may be suitable for pharmacological screens the inventors cultured iOL in the presence of compounds identified in earlier rodent studies^{1, 18-20}. In contrast to these earlier studies, some but not all of these compounds increased the number of O4⁺ iOL in a dose-dependent manner and were at least as effective as T3, a known promoter of oligodendroglial differentiation. Furthermore, only a subset of these drugs enhanced the maturation of O4⁺ iOL into MBP⁺ mature OL; suggesting that the compounds affect different stages of oligodendroglial differentiation. Miconazole demonstrated the strongest effect on iOL; this is in line with an earlier publication by Najm and colleagues in which they reported a strong differentiation promoting effect of miconazole on OL¹. However, a toxic effect with a fivefold higher concentration was observed, suggesting that miconazole might have a narrow range of efficacy. The inventors' observations thus suggest that there are species-specific differences between rodent and human OL that could be relevant for drug screens aiming at identifying compounds that promote oligodendroglial differentiation.

To determine whether iOL are suitable for disease modeling, the inventors characterized in a proof of concept study the phenotypes of iOL derived from a patient diagnosed with an inherited form of FTD. FTD is characterized by cortical degeneration of the frontal and temporal lobe that in 15 to 20% of patients with an inherited form of FTD is due to mutations in the MAPT gene that encodes the microtubule associated protein TAU located on chromosome 17q21. The neuropathology of FTDP-17 patients with mutations in the MAPT gene is characterized by TAU⁺ inclusions in neurons and glia including OL (for review see³⁸). Furthermore, extensive myelin pathology can be observed in patients with FTD²³⁻²⁵. In OL TAU regulates and stabilizes the microtubule network that is also involved in the transport of RNA granules, for examples those containing MBP mRNA. Knockdown of TAU or mutated TAU in rodent OL impairs process outgrowth and the differentiation into MBP⁺ myelinating mature OL^{39, 40}. Therefore the inventors assessed whether changes in OL may directly contribute to the white matter pathology observed in FTDP-17 patients. In iOL from patients with a N279K mutation in the MAPT gene, the inventors observed as expected, significantly increased expression levels of the 4R Tau isoform. Furthermore, the inventors observed an increased susceptibility to cell death induced by respiratory stress compared to gene corrected control cell lines, similar to that reported in iPSC-derived neurons from the same patient¹⁷. These data suggest that MAPT mutations in OL may directly contribute to myelin pathology and thus to disease progression in patients with FTDP-17.

The present invention demonstrates that SOX 10, and in particular a combination of three TFs, namely SOX10, OLIG2 and NKX6.2, greatly accelerates the generation of OL from iPSC-derived NPC and that these cells are suitable for disease modeling and pharmacological screens. Thus, the method according to the invention significantly facilitates the development of high-throughput screening platforms and the study of human myelin diseases and repair strategies using patient-derived iPSC.

The present invention is further characterized by the following items:
1. A method of generating oligodendroglial lineage cells, the method comprising the steps of:
(a) providing human cells selected from the group consisting of neural progenitor cells (NPCs), pluripotent stem cells (PSCs), induced pluripotent stem cells (iPSCs) and fibroblasts;
(b) inducing and/or increasing expression of the transcription factor SOX10, optionally in combination with OLIG2 and/or NKX6.2 in the cells;
(c) culturing the cells; thereby generating the oligodendroglial lineage cells.
2. The method of item 1, wherein the oligodendroglial lineage cells express one or more markers selected from the group consisting of PDGFRA, ST8SIA1, NG2, O4, GALC, O1, PLP, MBP, CNP, MAG, OLIG1, MOG, and a combination thereof.
3. The method of any one of the preceding items, wherein the NPCs are derived from PSCs or iPSCs.
4. The method of any one of the preceding items, wherein the expression of one or more of the transcription factors SOX10, OLIG2 and NKX6.2 in step (b) is increased compared to endogenous expression of the corresponding transcription factors.
5. The method of any one of the preceding items, wherein the expression of one or more of the transcription factors SOX10, OLIG2 and NKX6.2 is an ectopic expression.
6. The method of any one of the preceding items, wherein one or more nucleic acid(s) encoding one or more of the transcription factors SOX10, OLIG2 and NKX6.2 is/are introduced in the cells of step (a).
7. The method of the preceding item, wherein expression of one or more of the transcription factors SOX10, OLIG2 and NKX6.2 encoded by the one or more nucleic acid(s) is under the control of an inducible or constitutive promoter.
8. The method of the preceding item, wherein the promoter is inducible by tetracycline.
9. The method of any one of items 6-8, wherein the one or more nucleic acid(s) is/are introduced in the form of one or more viral or non-viral vectors.
10. The method of any one of items 6-9, wherein the one or more nucleic acid(s) is/are introduced in the form of one or more lentiviral vectors.
11. The method of any one of the preceding items, wherein, in step (c), the cells are cultured for a pre-determined amount of time following inducing and/or increasing expression.
12. The method of any one of the preceding items, wherein, in step (c), the cells are cultured for at least 7, 14, 21, 28 or 35 days following inducing and/or increasing expression.
13. The method of any one of the preceding items, wherein, after culturing the cells in step (c) for 7 days following inducing and/or increasing expression, at least 5%, preferably at least 6%, more preferably at least 7%, still more preferably at least 8% of the cells are O4⁺ oligodendroglial lineage cells.
14. The method of any one of the preceding items, wherein, after culturing the cells in step (c) for 14 days following inducing and/or increasing expression, at least 15%, preferably at least 16%, more preferably at least 17%, still more preferably at least 18% of the cells are O4⁺ oligodendroglial lineage cells.
15. The method of any one of the preceding items, wherein, after culturing the cells in step (c) for 21 days following inducing and/or increasing expression, at least 30%, preferably at least 33%, more preferably at least 36%, still more preferably at least 39% of the cells are O4⁺ oligodendroglial lineage cells.
16. The method of any one of the preceding items, wherein, after culturing the cells in step (c) for 28 days following inducing and/or increasing expression, at least 55%, preferably at least 59%, more preferably at least 63%, still more preferably at least 67% of the cells are O4⁺ oligodendroglial lineage cells.
17. The method of any one of the preceding items, wherein, after culturing the cells in step (c) for about 35 days following inducing and/or increasing expression, at least 20%, preferably at least 25%, more preferably at least 30%, still more preferably at least 35% of O4⁺ oligodendroglial lineage cells are also MBP⁺.
18. The method of the preceding item, wherein the MBP⁺ oligodendroglial lineage cells comprise a subpopulation of cells which are also positive for the mature oligodendroglial markers CNP and MAG.
19. The method of any one of the preceding items, wherein, in step (c), the cells are cultured in a first medium for about 2-4 days and thereafter in a second medium.
20. The method of the preceding item, wherein the second medium comprises a higher concentration of an inducer of oligodendroglial lineage differentiation than the first medium.
21. The method of the preceding item, wherein the inducer of oligodendroglial lineage differentiation is a thyroid hormone, miconazole or benztropine, preferably the thyroid hormone triiodothyronine (T3).
22. The method of any one of items 19-21, wherein the first medium and the second medium comprise DMEM-F12 comprising 0.1-10 mM glutamine and optionally 0.1-10% serum, and the second medium comprises an inducer of oligodendroglial lineage differentiation, preferably T3.
23. The method of any one of items 19-22, wherein the first medium further comprises one or more of N2 supplement, B27 supplement lacking vitamin A, penicillin/streptomycin, Smoothened agonist (SAG), Platelet-Derived Growth Factor (PDGF), Neurotrophin-3 (NT3), Insulin-like Growth Factor-I (IGF-I), ascorbic acid (AA), Trace Elements B, an inducer of oligodendroglial lineage differentiation, preferably T3, progesterone, putrescine, selenite, transferrin and/or insulin.
24. The method of any one of items 19-23, wherein the second medium further comprises one or more of N2 supplement, B27 supplement lacking vitamin A, penicillin/streptomycin, 1-100 ng/ml T3, NT3, IGF-I, AA, Trace Elements B and activators of protein kinase A such as dbcAMP.
25. The method of any one of the preceding items, wherein expression of GALC, OLIG1, MOG and/or MBP is upregulated in the generated oligodendroglial lineage cells compared to the cells provided in step (a).
26. The method of any one of the preceding items, wherein expression of SOX1, PAX6 and/or PAX7 is downregulated in the generated oligodendroglial lineage cells compared to the cells provided in step (a).
27. The method of any one of the preceding items, wherein the generated oligodendroglial lineage cells express PDGFRA and/or ST8SIA1.
28. The method of any one of the preceding items, wherein the generated oligodendroglial lineage cells are capable of producing myelin-like sheaths surrounding axons of co-cultured iPSC-derived neurons in an in vitro assay.
29. The method of any one of the preceding items, wherein the generated oligodendroglial lineage cells are capable of remyelinating demyelinated axons in a Shi/Shi Rag2^{-/-} mouse model.
30. An oligodendroglial lineage cell obtainable by the method of any one of the preceding items.
31. An oligodendroglial lineage cell according to item 30, wherein the cell is O4⁺ and/or MBP⁺.
32. A recombinant vector comprising a nucleotide sequence encoding SOX10, OLIG2 and NKX6.2.
33. The recombinant vector of the preceding item, wherein the vector is a non-viral vector or a viral vector.
34. The recombinant vector of any one of items 32-33, wherein the vector is a retroviral vector, preferably a lentiviral vector.
35. The recombinant vector of any one of items 32-34, wherein the vector is suitable for transduction of a human cell.
36. A human NPC, PSC, iPSC or fibroblast comprising one or more exogenous nucleic acid(s) encoding at least one or more of SOX10, OLIG2 and NKX6.2.
37. The cell of the preceding item, wherein the one or more nucleic acid(s) encode(s) SOX10 and OLIG2.
38. The cell of any one of items 36-37, wherein the one or more nucleic acid(s) encode(s) SOX10, OLIG2 and NKX6.2.
39. The cell of any one of items 36-38, wherein the one or more exogenous nucleic acid(s) comprise(s) an inducible promoter which controls expression of at least one of SOX10, OLIG2 and NKX6.2.
40. The cell of item 39, wherein the promoter is inducible by tetracycline.
41. A method of screening for a compound promoting oligodendroglial differentiation and/or maturation, the method comprising the steps of:
(a) providing human cells selected from the group consisting of NPCs, PSCs, iPSCs and fibroblasts or providing cells according to any one of items 36-40;
(b) inducing and/or increasing expression of the transcription factor SOX10, optionally in combination with OLIG2 and/or NKX6.2 in the cells;
(c) culturing the cells for a pre-determined amount of time following inducing and/or increasing expression, wherein a first sample of the cells is cultured in the presence of a compound to be tested and a second sample of the cells is cultured in the absence of the compound;
(d) determining the percentage of cells which are positive for a marker of an oligodendrocyte developmental stage in the first sample and in the second sample;
   wherein a higher percentage of cells which are positive for the marker in the first sample than in the second sample indicates that the compound promotes oligodendroglial differentiation and/or maturation.
42. The method of screening of item 41, wherein the compound is a candidate agent for treating neurodegenerative and/or myelin diseases.
43. The method of screening of any one of items 41-42, wherein the marker is a marker for one or more of oligodendrocyte precursor cells (OPCs), differentiated oligodendrocytes, mature oligodendrocytes and/or myelinating oligodendrocytes.
44. The method of screening of any one of items 41-43, wherein the marker is selected from the group consisting of PDGFRA, ST8SIA1, NG2, O4, GALC, O1, PLP, MBP, CNP, MAG, OLIG1, MOG, and a combination thereof.
45. The method of screening of any one of items 41-44, wherein the screening method is a high throughput screening.
46. A use of oligodendroglial lineage cells obtainable by the method of any one of items 1-29 or of a cell of any one of items 36-40 in a screening method, preferably wherein the screening method is a high throughput screening, or in expression profiling or in disease modeling.
47. A pharmaceutical composition comprising cells obtainable by the method of any one of items 1-29 and/or comprising cells of any one of items 36-40.
48. The pharmaceutical composition of item 47, the cell obtainable by the method of any one of items 1-29, or the cell of any one of items 36-40 for use as a medicament.

| SEQ | | |
|---|---|---|
| ID | Descr. | Sequence |
| 7 | SOX | MAEEQDLSEVELSPVGSEEPRCLSPGSAPSLGPDGGGGGS |
| | 10 | GLRASPGPGELGKVKKEQQDGEADDDKFPVCIREAVSQVL |
| | pro- | SGYDWTLVPMPVRVNGASKSKPHVKRPMNAFMVWAQAARR |
| | tein | KLADQYPHLHNAELSKTLGKLWRLLNESDKRPFIEEAERL |
| | | RMQHKKDHPDYKYQPRRRKNGKAAQGEAECPGGEAEQGGT |
| | | AAIQAHYKSAHLDHRHPGEGSPMSDGNPEHPSGQSHGPPT |
| | | PPTTPKTELQSGKADPKRDGRSMGEGGKPHIDFGNVDIGE |
| | | ISHEVMSNMETFDVAELDQYLPPNGHPGHVSSYSAAGYGL |
| | | GSALAVASGHSAWISKPPGVALPTVSPPGVDAKAQVKTET |
| | | AGPQGPPHYTDQPSTSQIAYTSLSLPHYGSAFPSISRPQF |
| | | DYSDHQPSGPYYGHSGQASGLYSAFSYMGPSQRPLYTAIS |
| | | DPSPSGPQSHSPTHWEQPVYTTLSRP |
| 8 | OLIG2 | MDSDASLVSSRPSSPEPDDLFLPARSKGSSGSAFTGGTVS |
| | pro- | SSTPSDCPPELSAELRGAMGSAGAHPGDKLGGSGFKSSSS |
| | tein | STSSSTSSAAASSTKKDKKQMTEPELQQLRLKINSRERKR |
| | | MHDLNIAMDGLREVMPYAHGPSVRKLSKIATLLLARNYIL |
| | | MLTNSLEEMKRLVSEIYGGHHAGFHPSACGGLAHSAPLPA |
| | | ATAHPAAAAHAAHHPAVHHPILPPAAAAAAAAAAAAAVSS |
| | | ASLPGSGLPSVGSIRPPHGLLKSPSAAAAAPLGGGGGGSG |
| | | ASGGFQHWGGMPCPCSMCQVPPPHHHVSAMGAGSLPRLTS |
| | | DAK |
| 9 | NKX- | MDTNRPGAFVLSSAPLAALHNMAEMKTSLFPYALQGPAGF |
| | 6.2 | KAPALGGLGAQLPLGTPHGISDILGRPVGAAGGGLLGGLP |
| | pro- | RLNGLASSAGVYFGPAAAVARGYPKPLAELPGRPPIFWPG |
| | tein | VVQGAPWRDPRLAGPAPAGGVLDKDGKKKHSRPTFSGQQI |
| | | FALEKTFEQTKYLAGPERARLAYSLGMTESQVKVWFQNRR |
| | | TKWRKRHAAEMASAKKKQDSDAEKLKVGGSDAEDDDEYNR |
| | | PLDPNSDDEKITRLLKKHKPSNLALVSPCGGGAGDAL |

The Examples illustrate the invention:

### EXAMPLE 1

### Methods

### Culturing of Human PSC

The iPSC included in this study have previously been generated and characterized^{13, 17} iPSC colonies were maintained on a layer of mitotically inactivated MEFs in human ESC medium consisting of Knockout DMEM (Invitrogen) with 20% Knockout Serum Replacement (Invitrogen), 1 mM beta-mercaptoethanol (Invitrogen), 1% nonessential amino acids (NEAA, Invitrogen), 1% penicillin/streptomycin/glutamine (PAA), freshly supplemented with 5 ng/mL FGF2 (Peprotech). PSC were split at ratios of 1:6 to 1:8 every seven days by mechanic disaggregation with 1 mg/mL collagenase IV (Invitrogen). The work with the human ESC line HUES6 was approved by the Robert-Koch-Institute, Berlin, Germany.

### Generation and Culturing of Human NPC

NPC were derived from human PSC by treatment with small molecules as previously described^{13, 17}. In short, PSC colonies from passages 10-15 were mechanically sectioned and enzymatically detached from MEFs. Pieces of PSC colonies were collected by sedimentation, resuspended in ESC medium (without FGF) supplemented with 10 μM SB-431542 (Ascent Scientific), 1 μM dorsomorphin (Tocris), 3 μM CHIR99021 (CHIR; Axon Medchem) and 0.5 μM purmorphamine (PMA; Alexis) and subsequently cultured as embryoid bodies (EBs) in petri dishes. The medium was changed after two days to N2B27 medium consisting in equal parts of DMEM-F12 (Invitrogen) and Neurobasal (Invitrogen) with 1:200 N2 supplement (Invitrogen), 1:100 B27 supplement lacking vitamin A (Invitrogen), 1% penicillin/streptomycin/glutamine and with the same small molecule supplements as mentioned afore. On day 4, SB-431542 and dorsomorphin were withdrawn and 150 μM ascorbic acid (AA; Sigma) was added to the medium. On day 6, EBs were disintegrated into smaller pieces and plated on matrigel-coated (Matrigel, growth factor reduced, high concentration; BD Biosciences) 12-well plates (Nunc) in NPC expansion medium (NPCM) consisting of N2B27 medium supplemented with 3 μM CHIR, 0.5 μM SAG (Cayman Chemical) and 150 μM AA. Cells were split once a week at ratios of 1:15 to 1:20 by treatment with accutase (Sigma). Regular tests for mycoplasma contamination using the MycoAlert mycoplasma detection kit (Lonza) were negative.

### Lentiviral Vector Construction and Production of Lentiviral Particles

The coding regions of SOX10, OLIG2, ASCL1, NKX2.2, NKX6.1, NKX6.2, MYT1 and RFP were amplified by PCR, validated by sequencing, cloned into pCR8/GW/TOPO (Invitrogen) according to the manufacturer's instruction, and recombined into pLV-tetO-attR1/R2⁴¹ by LR clonase II (Invitrogen).

To construct the polycistronic lentiviral SON vector, we used a third generation lentiviral vector, which we further equipped with the retroviral SFFV (spleen focus forming virus) U3 promoter and the reprogramming cassette⁴². To be optionally able to excise the reprogramming cassette later, we incorporated a FRT (Flp recognition target site) in the 3′ U3 region. The human cDNAs encoding SOX10, OLIG2 and NKX6.2 were inserted to create a 3-in-1 vector, in which the transcription factor genes are co-expressed and linked by 2A self-cleavage sites (P2A, T2A). Furthermore, we introduced an IRES-RFP cassette (encoding the dimeric RFP variant dTomato) for visualization of vector expression as previously described⁴² (FIG. 12). The construct was validated by sequencing. Lentiviral particles were produced by co-transfection of 293T cells with individual expression vectors in combination with the packaging plasmids psPAX2 (Addgene #12260) and pMD2.G (Addgene #12259). Virus containing supernatants were harvested at 48 and 72 h post-transfection and filtered with 0.45 μm PVDF membrane (Millipore). Viral particles were subsequently concentrated by ultracentrifugation, resuspended in N2B27 medium and stored at −80° C.

Transduction of NPC for TF Screening Human NPC were seeded with a density of 1×10⁵ cells/well in 12-well plates, allowed to attach overnight and transduced with equal volumes of concentrated Lenti-rtTA and 1-TF virus particle supplemented with 5 μg/ml protamine sulfate (Sigma) in fresh NPCM. 2-TF infections were done by mixing equivalent volumes of Lenti-rtTA, pLV-TetO-SOX10 and 1-TF virus particle for infection. For 3-TF infections, the volume of each virus was reduced by one quarter and equivalent volumes of Lenti-rtTA, pLV-TetO-SOX10, pLV-TetO-OLIG2 and 1-TF were mixed for NPC transduction. Viral medium was removed after 24 h and replaced by N2B27 medium supplemented with 1 μM SAG, 10 ng/ml PDGF (Peprotech), 10 ng/ml NT3 (Peprotech), 10 ng/ml IGF-I (Peprotech), 200 μM AA, 1:1000 Trace Elements B (Corning), 60 ng/ml Triiodo-L-Thyronine (T3; Sigma). The end of the virus infection period was termed day 0 and transgene expression was induced with 2 μg/ml doxycycline (Clontech) for 14 days. Medium was replaced every other day and cells were fixed in 4% paraformaldehyde (PFA; Sigma) in PBS (Invitrogen) for ICC analysis at day 14 of differentiation. Investigators were blinded for ICC analysis.

### Oligodendroglial Differentiation

For oligodendroglial differentiation, human NPC were seeded with a density of 1×10⁵ cells/well in 12-well plates, allowed to attach overnight and transduced with concentrated SON lentiviral particle and 5 μg/ml protamine sulfate in fresh NPCM. Viral medium was removed after 24 h and replaced with glial induction medium (GIM) consisting of DMEM-F12 with 1:200 N2 supplement, 1:100 B27 supplement lacking vitamin A, 1% penicillin/streptomycin/glutamine, 1 μM SAG, 10 ng/ml PDGF, 10 ng/ml NT3, 10 ng/ml IGF-I, 200 μM AA, 1:1000 Trace Elements B, 10 ng/ml T3. The end of the virus infection period was termed day 0. After 4 days, GIM was replaced by differentiation medium (DM) consisting of DMEM-F12 with 1:200 N2 supplement, 1:100 B27 supplement lacking vitamin A, 1% penicillin/streptomycin/glutamine, 60 ng/ml T3, 10 ng/ml NT3, 10 ng/ml IGF-I, 200 μM AA, 1:1000 Trace Elements B and 100 μM dbcAMP (Sigma). After 7 to 10 days of differentiation, cells were detached and singularized by treatment with accutase and reseeded at densities of 2.5×10⁵ cells in 12-well plates and 1.5×10⁵ cells in 24-well plates containing glass coverslips.

### Immunocytochemistry

For immunocytochemical analysis, an equal volume of 4% PFA in PBS was added to the culture medium and cells were pre-fixed for 10 min at room temperature (RT). After removing the supernatant, cells were fixed for an additional 15 minutes with 4% PFA in PBS and washed three times with PBS. Fixed cells were permeabilized by adding 0.2% Triton X-100 (Sigma) in PBS for 15 min at RT (This step was omitted for NG2, O4 and GalC staining). Subsequently, blocking was performed by incubating the cells with 5% normal goat serum (NGS; Gibco) and 5% fetal calf serum (FCS; Gibco) for 30 min at RT. Primary antibodies were applied overnight at 4° C. in blocking solution. Following three washing steps with PBS, cells were incubated with Alexa Fluor conjugated secondary antibodies diluted in PBS for 1 h at RT. Cells were subsequently washed three times with PBS, including a Hoechst counterstaining for nuclei in the second washing step. Cells on glass coverslips were mounted in Dako Fluorescent Mounting Medium (Dako) and visualized on a Zeiss LSM700 confocal microscope. Cells on plastic cell culture plates were visualized on a Leica DM16000 B inverted microscope. Primary antibodies used in this study are listed in Table 1.

TABLE 1
Primary Antibodies for immunocytochemistry

| Antibody | Dilution | Company |
|---|---|---|
| mouse anti-NESTIN | 1:300 | R&D (MAB1259) |
| goat anti-SOX1 | 1:150 | R&D (AF3369) |
| rabbit anti-NG2 | 1:200 | Millipore (AB5320) |
| mouse anti-O4 | 1:1000 | R&D (MAB1326) |
| mouse anti-GALC | 1:100 | Millipore (MAB342) |
| rat anti-MBP | 1:50 | Abcam (AB7349) |
| rabbit anti-Ki67 (SP6) | 1:250 | Abcam (AB16667) |
| mouse anti-CNP | 1:250 | BioLegend (836401) |
| anti-MAG | 1:400 | Abcam (AB89780) |
| mouse anti-TUBBIII (TUJ1) | 1:750 | Covance (MMS-435P) |
| mouse anti-AT8 | 1:150 | Innogenetics (90206) |

### Flow Cytometry Analysis

Cells were enzymatically detached and singularized by accutase treatment for 10 minutes at 37° C. Following washing with PBS, singularized cells were re-suspended in PBS/0.5% BSA buffer and filtered through a 70 μm cell strainer (BD Falcon). After determination of cell number, cells were incubated with mouse IgM anti-O4-APC antibody (Miltenyi Biotec) following the manufacturer's protocol. Stained cells were washed, resuspended in PBS/0.5% BSA buffer (5×10⁶ cells/ml) and immediately sorted using a FACSAria cell sorter (BD Biosciences). Debris, doublets and aggregates were excluded by appropriate gating using forward and side scatter. Additionally, DAPI was used for dead cell exclusion. Unstained cells were used to set background fluorescence and undifferentiated human NPC were used as negative controls.

### Neuronal Differentiation

Human iPSC-derived NPC were differentiated into neurons as previously described¹⁷. Briefly, iPSC-derived NPC were cultured with N2B27 medium supplemented with 1 μM SAG (Cayman Chemical), 2 ng/ml BDNF (Peprotech), 2 ng/ml GDNF (Peprotech) and 100 μM AA (Sigma) for 6 days and afterwards with N2B27 medium supplemented with 2 ng/ml BDNF (Peprotech), 2 ng/ml GDNF (Peprotech), 0.5 ng/ml TGF-β3 (Peprotech), 100 μM dbcAMP and 100 μM AA. Additionally, 5 ng/ml Activin A (Sigma) was added to the medium from day 7 to 9. After 9 days of neuronal differentiation, cells were detached, singularized by treatment with accutase and reseeded at densities of 2×10⁵/well neurons in 24-well plates containing glass coverslips. After 21 days of differentiation, cells were used for co-culture experiments.

### In Vitro Myelination Assay and 3D Culture

To assess the in vitro myelination capacity of iOLs, O4⁺ cells were purified at differentiation day 21 by magnetic cell sorting using anti-O4 MicroBeads (Miltenyi Biotec) following the manufacturer's protocol. Purified iOLs were added to 21 day old neuronal cultures derived from iPSC-derived NPC populations with the aforementioned protocol at densities of 1×10⁵ cells per well in matrigel-coated 24-well plates containing glass coverslips. Co-cultures were maintained in DM supplemented with 2 ng/ml BDNF and 2 ng/ml GDNF. After 14 to 28 days of co-culture, cells were fixed in 4% PFA for immunocytochemical analysis.

For the 3D culture experiments, nanofiber chamber slides (Nanofiber Solutions) containing aligned nanofiber polymers were pre-coated with 10 μg/ml laminin (Sigma) and incubated with DM supplemented with 2 ng/ml BDNF and 2 ng/ml GDNF at 37° C. over night. O4⁺ iOLs were purified after 21 days of differentiation using MACS and were reseeded at a density of 5×10⁴ cells per chamber in DM supplemented with 2 ng/ml BDNF and 2 ng/ml GDNF. Half of the medium was changed every other day and cells were fixed in 4% PFA after 14 days for immunocytochemical analysis.

All experiments have been successfully repeated with at least three biological independent iOL populations.

### Isolation of Primary Adult Human OL

Brain tissue was obtained from adults undergoing surgical resections as treatment for non-tumor-related intractable epilepsy in accordance with the guidelines set by the Biomedical Ethics Unit of McGill University. As described⁴³ tissue specimens were enzymatically digested and placed on a linear 30% Percoll density gradient (Pharmacia Biotech, Piscataway, N.J.). Microglia were separated and removed by an initial adhesion step in which the total cell fraction was cultured for 24 hours in non-coated flasks. The floating cell fraction was subjected to immunomagnetic bead selection with the A2B5 antibody (IgM) to select out progenitor cells⁴³. The non-selected fraction (referred to as primary human oligodendrocytes (pOL)) was plated on poly-L-lysine coated glass chamber slides in defined medium (DFM) consisting of Dulbecco's modified essential medium DMEM-F12 supplemented with N1 (Sigma), 0.01% bovine serum albumin (BSA), 1% penicillin-streptomycin and B27 supplement lacking Vitamin A, 10 ng/ml PDGF, 10 ng/ml bFGF (Sigma) and 2 nM T3. After six days in DFM cells were either lysed Isol-RNA Lysis Reagent (Thermo Fisher) or fixed in 4% PFA for immunocytochemical analysis demonstrating that ˜90% of cells were O4⁺ and MBP⁺ (FIG. 8). Expression of O1 (galactocerbroside) has been documented on these cells by flow cytometry.

### Whole Genome Expression Analysis

Total RNA was quantified using a NanoDrop Spectrophotometer ND-1000 (NanoDrop Technologies, Inc.) and its integrity was assessed using a 2100 Bioanalyzer (Agilent Technologies). 5 ng of total RNA was used as input for cRNA synthesis with the Genechip WT Pico Reagent Kit (Affymetrix) according to the manufacturer's protocol. The processed human iPSC-derived samples were hybridized onto GeneChip Human Transcriptome 2.0 Arrays (Affymetrix) and the primary human samples were hybridized onto Human Gene 2.0 ST Arrays (Affymetrix) for 16 hours following the manufacturer's protocol. Next, GeneChips were washed and stained using the GeneChip Hybridization, Wash and Stain Kit (Affymetrix) and the GeneChip Fluidics Station 450 (Affymetrix). The Arrays were scanned by the GeneChip Scanner 3000 7G (Affymetrix) and first data processing was performed by the GeneChip Command Console Viewer 3.2 (Affymetrix). iPSC-derived NPC and iOL RNA samples were processed at University Hospital Muenster, pOL samples were processed by McGill University and Genome Quebec Innovation Centre.

### Microarray Data Processing

Gene expression data for iPSC samples were obtained from Gene Expression Omnibus (GSE61358) and used as a negative control. All microarray data from iPSC, pOL, iPSCderived NPC and iOL samples were processed using Bioconductor package ‘oligo’⁴⁴. Background subtraction, quantile gene expression normalization and summarization were performed using robust multi-array average method implemented in the ‘oligo’ package. Variance stabilization was performed using the log 2 scaling. Differentially expressed genes among iOL and iPSC-derived NPC samples were identified through an unpaired one-way between subject ANOVA. p values were corrected for multiplicity according to the Benjamini-Hochberg procedure with a threshold of 0.05 (false discovery rate [FDR]). Results were further filtered by fold change magnitude (|fold change|≥2). Graphics were obtained using ‘ComplexHeatmap’ and ‘VennDiagram’ R-packages. Hierarchical cluster of samples was performed with ‘pvclust’ R-package using the one minus the sample correlation metric and complete-linkage clustering method. Probe mapping to the corresponding gene information was performed using Bioconductor package ‘Annotationdbi’.

### Cell Transplantation

Shiverer mice were crossed to Rag2 null immunodeficient mice to generate a line of Shi/Shi Rag2^{-/-} dysmyelinating immunodeficient mice. Mice were housed under standard conditions of 12-hour light/12-hour dark cycles with ad libitum access to dry food and water cycle at ICM animal facility. Experiments were performed according to European Community regulations and Inserm ethical committee and were approved by the local Darwin ethical committee.

To assay iOL contribution to developmental myelination, newborn pups (n=7) were cryoanesthetized before bilateral transplantation of 2×100,000 cells, rostral to the corpus callosum. Injections (10⁵ cells/μl) were performed 1 mm caudally, 1 mm laterally from the bregma and to a depth of 1 mm⁴⁶. Animals were sacrificed at 16 wpg for immunohistological studies (n=4) and electron microscopy (n=3).

To assay iOL involvement in remyelination, mice (n=4) of 8-9 weeks of age, were anaesthetized by intraperitoneal injection of a mixture of 100 mg/kg Ketamine (Alcyon) and 10 mg/kg Xylazine (Alcyon). Focal demyelination was performed as previously described³⁷ by stereotaxic injection of 1 μl of 1% LPC (Sigma-Aldrich) in 1% PBS into the dorsal funiculus of the spinal cord at the level of the 13th thoracic vertebrae. Forty-eight hours after demyelination, mice received a single injection (1 μl, 10⁵ cells/μl) of iOL at the site of demyelination. All injections (LPC or cells) were performed at low speed (1 μl/2 min) using a stereotaxic frame equipped with a micromanipulator and a Hamilton syringe. Animals were sacrificed at 12 wpg for immunolhistological studies.

### Immunohistochemistry

For immunohistochemistry, mice were sacrificed by transcardiac perfusion-fixation with 4% PFA in PBS and processed for freezing. Sagittal brain- and spinal cord cross sections of 12 μm thickness were performed with a cryostat (CM30505; Leica). In vivo characterization of grafted cells was performed by immunostaining using the following antibodies: anti-human cytoplasm (STEM121; SC Proven, 1:500) anti human nuclei (STEM101; SC Proven, 1:100) anti-MBP (Chemicon, AB980, 1:400), anti-MOG (mouse IgG1 hybridoma, clone C18C5; 1:20). Identification of neurofilaments was performed by anti-NF200 (N0142, Sigma-Aldrich, 1:200) or anti-NF165 (mouse IgG1 hybridoma 2H3, 1:20). Nodes of Ranvier were detected by anti-CASPR (1:1,000). For MBP staining sections were pre-treated with ethanol. For CASPR staining, slices were incubated in methanol (10 min at 20° C.) and saturated in the presence of 0.1% glycine (Research Organics). Secondary antibodies conjugated with FITC, TRITC (SouthernBiotech) or Alexa Fluor 647 (Life Technologies) were used respectively at 1:100 and 1:1,000. Nuclei were stained with Dapi (1 μg/ml, Sigma-Aldrich) (1:1,000). Tissue scanning, cell visualization and imaging were performed with a Carl Zeiss microscope equipped with ApoTome.2.

### Electron Microscopy

For electron microscopy, Shi/Shi Rag2^{-/-} mice were perfused with 1% PBS followed by a mixture of 4% paraformaldehyde/5% glutaraldehyde (Electron Microscopy Science) in 1% PBS. After 2 h post-fixation in the same solution, brains were cut in 100 μm-thick sections and fixed in 2% osmium tetroxide (Sigma-Aldrich) overnight. After dehydration, samples were embedded in Epon. Ultra-thin sections (80 nm) were examined with a HITACHI 120 kV HT-7700 electron microscope

### Compound Screen

To assess the sensitivity of iOL towards differentiation promoting drugs, iPSC-derived NPC were transduced with concentrated SON virus particle as described above. Viral medium was removed after 24 h and replaced with GIM lacking T3. The end of the virus infection period was termed day 0. At day 5 of differentiation, cells were detached, singularized and reseeded at densities of 5×10⁴ cells in matrigel-coated 48-well plates or 1.5×10⁵ cells in 24-well plates containing matrigel-coated glass coverslips. Cells were allowed to recover in minimum DM consisting of DMEM-F12 with 1:200 N2 supplement, 1:100 B27 supplement lacking vitamin A, 1% penicillin/streptomycin/glutamine, 200 μM AA and 100 μM dbcAMP. After 24 h cells were treated with vehicle alone (0.01% (v/v) DMSO) as a negative control, 60 ng/ml T3 as a positive control, or with a drug candidate dissolved in DMSO at three different concentrations (0.5 μM, 1 μM, 5 μM) in minimum DM. The drug candidates comprised miconazole (Sigma), clobetasol (TCI), benztropine (Sigma), indometacin (Sigma), clemastine (Sigma) and oxybutynin (Sigma). Medium was changed every other day and cells were fixed in 4% PFA for immunocytochemical analysis after 21 days of treatment. Investigators were blinded for immunocytochemical analysis.

### Generation and Characterization of N297K MAPT Neural Progenitor Cells and Isogenic Controls

The N279K MAPT iPSC-derived NPC included in this study have previously been generated and characterized¹⁷. Frozen NPC, termed FTDP-17-1-I and FDTP-17-1-II in the aforementioned publication were thawed at passages 10-14 and designated as MAPT-1 and MAPT-2 in this study.

### Quantitative RT-PCR

Total RNA was isolated from cell lysates using the RNeasy mini kit (QIAGEN) according to the manufacturer's protocol and including an on-column DNase digest (RNase free DNase Set; Qiagen). Quantification of total RNA was performed with Nanodrop ND1000 (Peqlab). cDNA was generated from isolated RNAs by reverse transcription using the High Capacity cDNA reverse Transcription Kit (Applied Biosystems). Quantitative RT-PCR was performed on an Applied Biosystems StepOne Plus real time cycler (Applied Biosystems) with the Power SYBR Green PCR master mix (Applied Biosystems). Specificity of the primers used for RT-PCR reactions was determined beforehand, by agarose gel electrophoresis. The melting curve of each sample was determined to ensure the specificity of the products. The quantitative RT-PCR conditions were 2 min at 50° C., 10 min at 95° C., 40 cycles of 15 sec at 95° C. and 1 min at 60° C. Relative expression levels were calculated using the 2^{-ΔΔct} method and normalized to biological reference samples and using GAPDH as the housekeeping gene unless otherwise noted. The primer sequences used in this study are listed in Table 2.

TABLE 2
Primer for quantitative RT-PCR

| | |
|---|---|
| hGAPDH_for | CTG GTA AAG TGG ATA TTG TTG CCA T |
| | (SEQ ID NO: 1) |
| hGAPDH_rev | TGG AAT CAT ATT GGA ACA TGT AAA CC |
| | (SEQ ID NO: 2) |
| hMAPT_total_for | CTC GCA TGG TCA GTA AAA GCA A |
| | (SEQ ID NO: 3) |
| hMAPT_total_rev | GGG TTT TTG CTG GAA TCC TGG T |
| | (SEQ ID NO: 4) |
| hMAPT_Exon10_for | CCA AGT GTG GCT CAA AGG AT |
| | (SEQ ID NO: 5) |
| hMAPT_Exon12_rev | CCC AAT CTT CGA CTG GAC TC |
| | (SEQ ID NO: 6) |

### Stress-Induced Cell Death

To examine the effect of rotenone (Sigma) on the viability of iOL derived from either N279K MAPT or Ctrl cells O4⁺ iOL were purified using the MACS technology after 21 days of differentiation and replated at a density of 8×10³ cells per well into matrigel-coated 96-well plates. After another 6 days in DM, cells were treated with either vehicle (0.01% (v/v) DMSO) or rotenone dissolved in DMSO at three different concentrations (100 nM, 250 nM, 500 nM). After 24 h, cells were fixed and cell toxicity was determined by immunocytochemical double-staining using antibodies against cleaved CASPASE 3 and O4. Investigators were blinded for immunocytochemical analysis.

### Statistics

Data of at least three independent differentiation experiments are presented as mean+SD. Statistical significance was determined by Student's t test and with One-way ANOVA, respectively.

### Accession Numbers

Microarray data have been deposited with Gene Expression Omnibus accession number GSE79914.

### EXAMPLE 2

### Identification of OL Lineage Inducing TFs in Human NPC

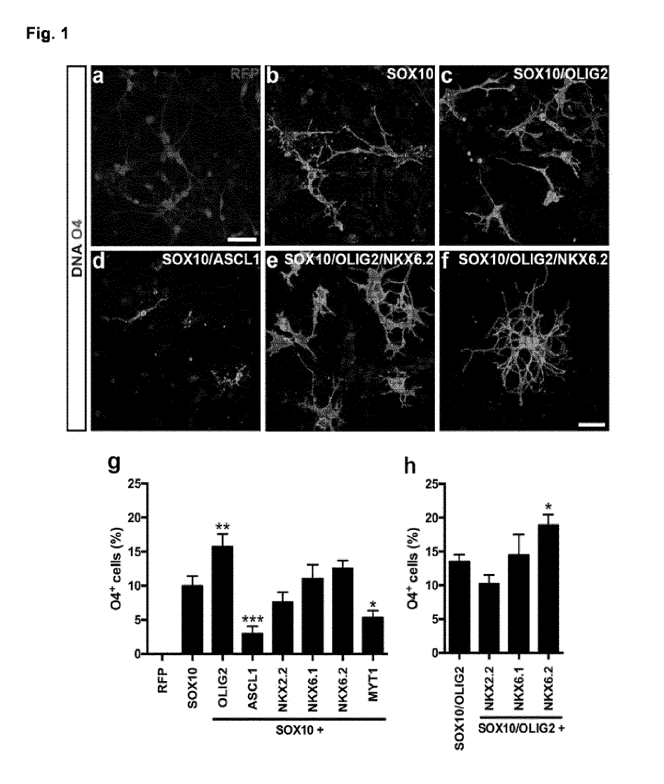

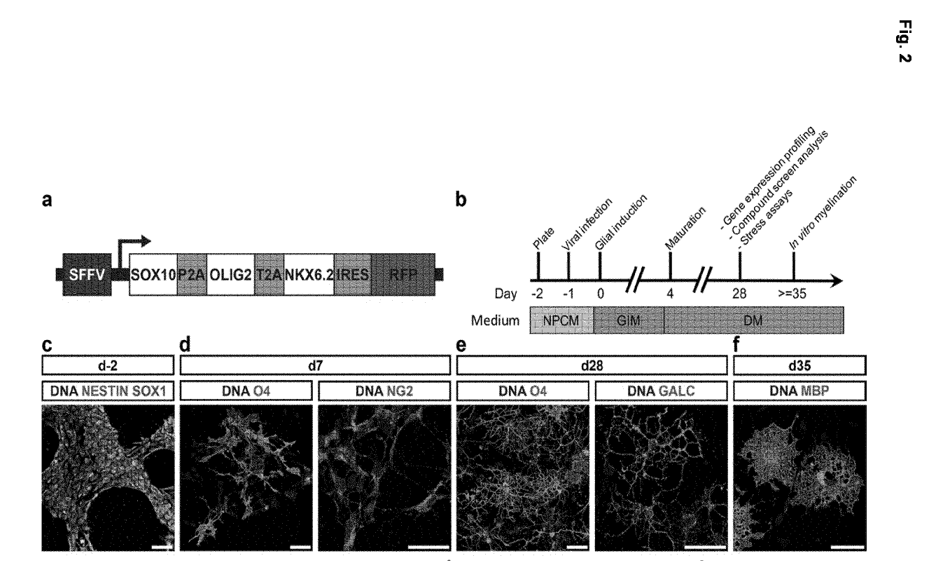

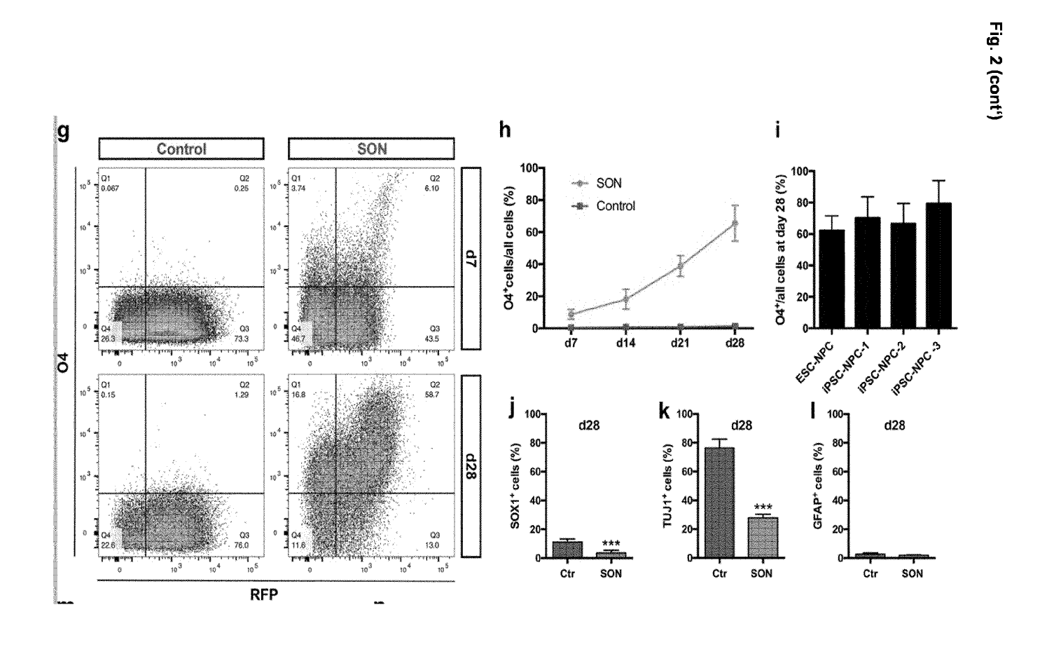

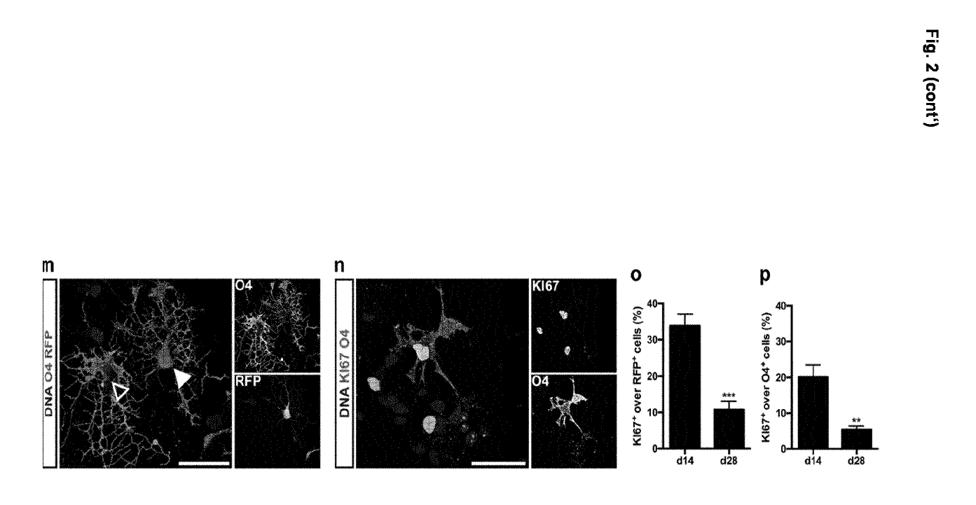

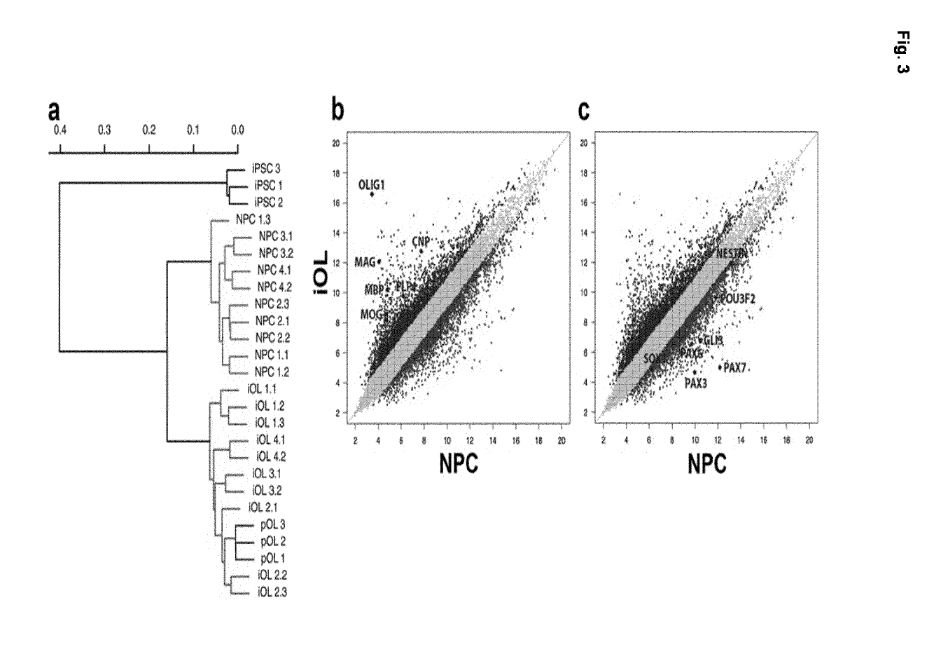

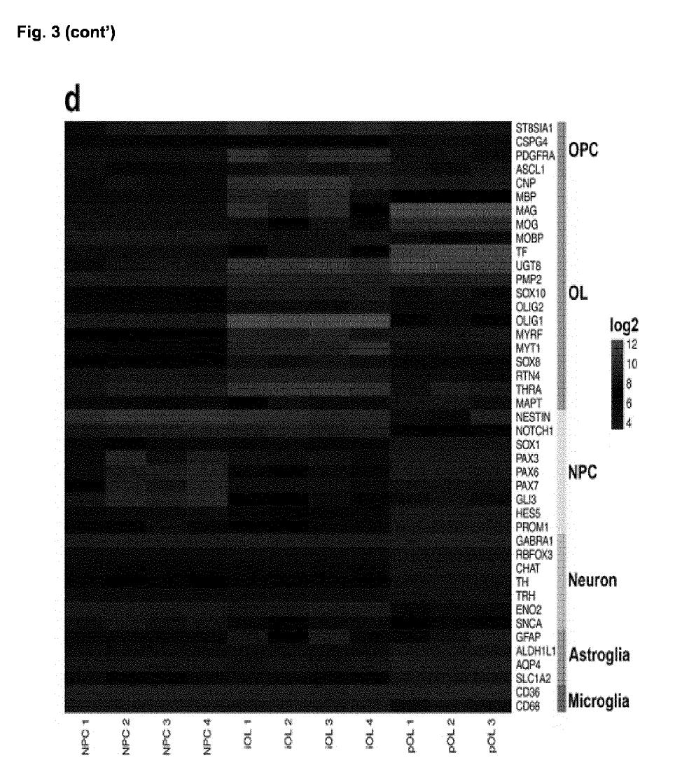

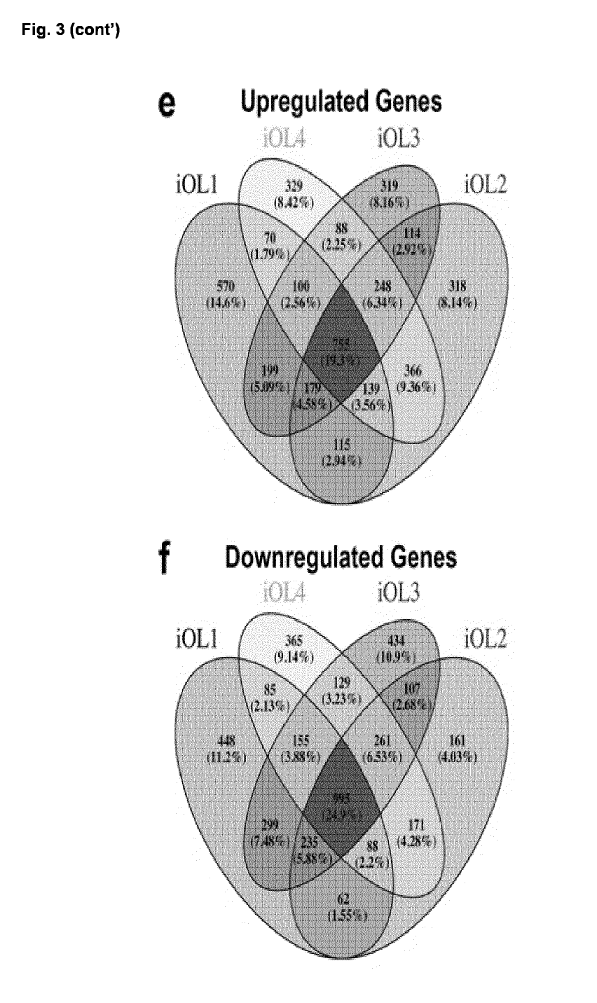

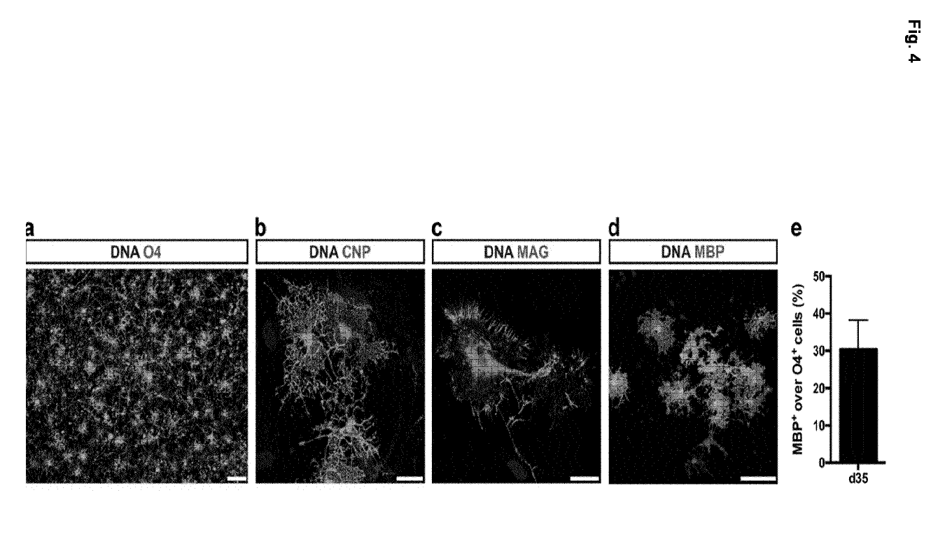

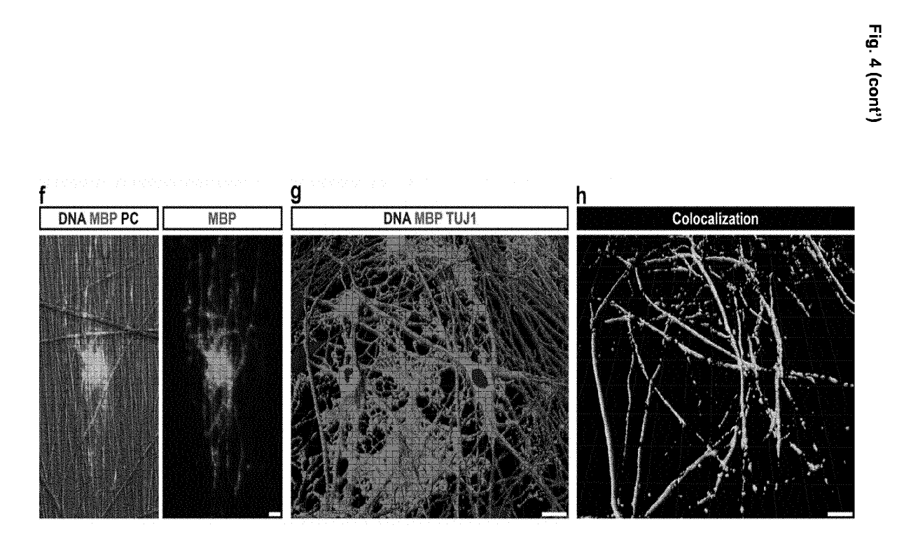

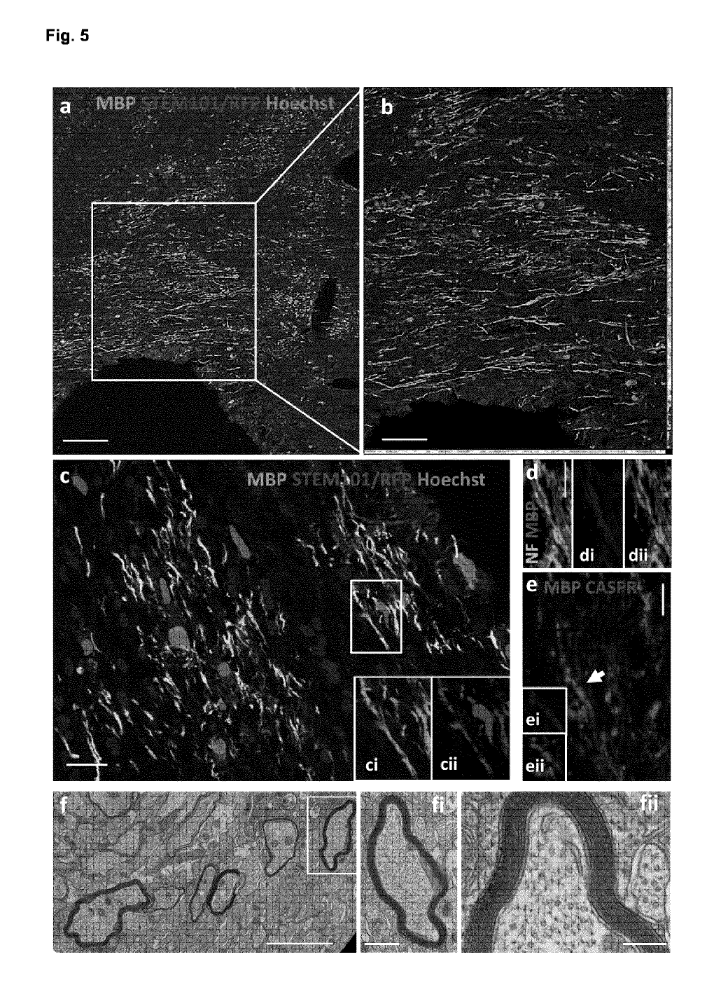

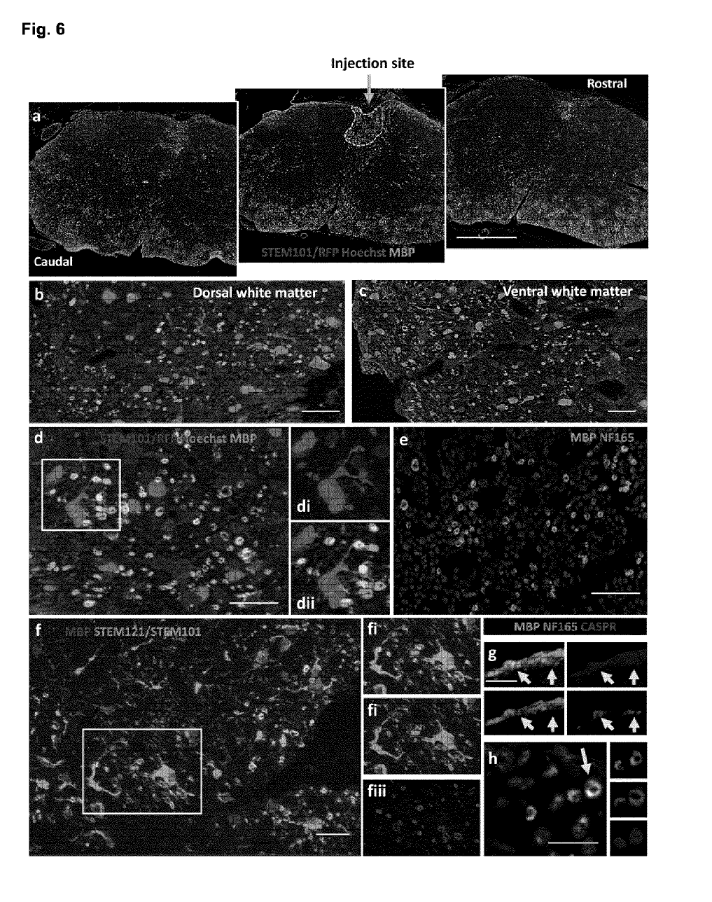

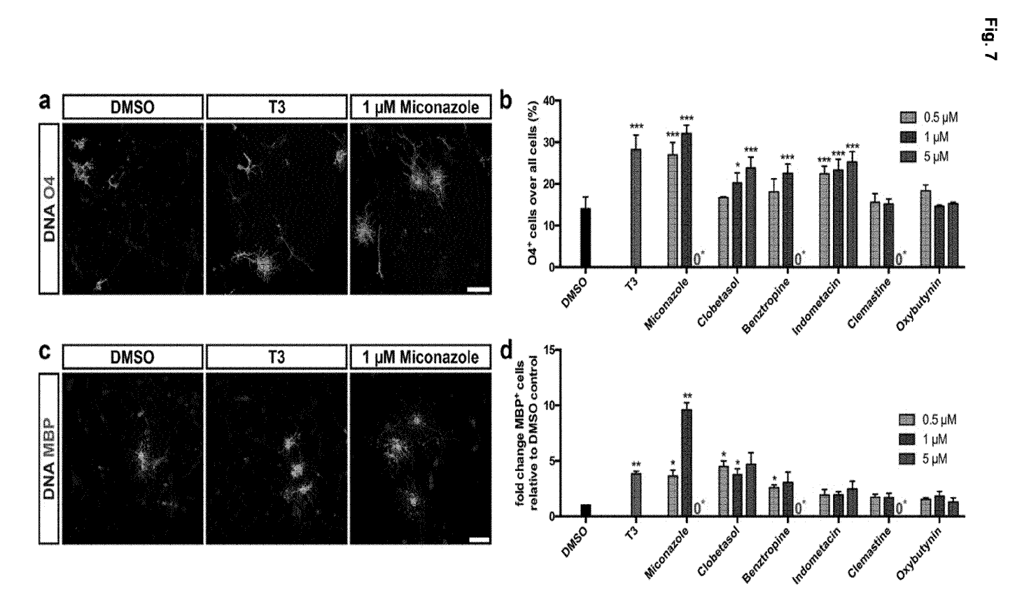

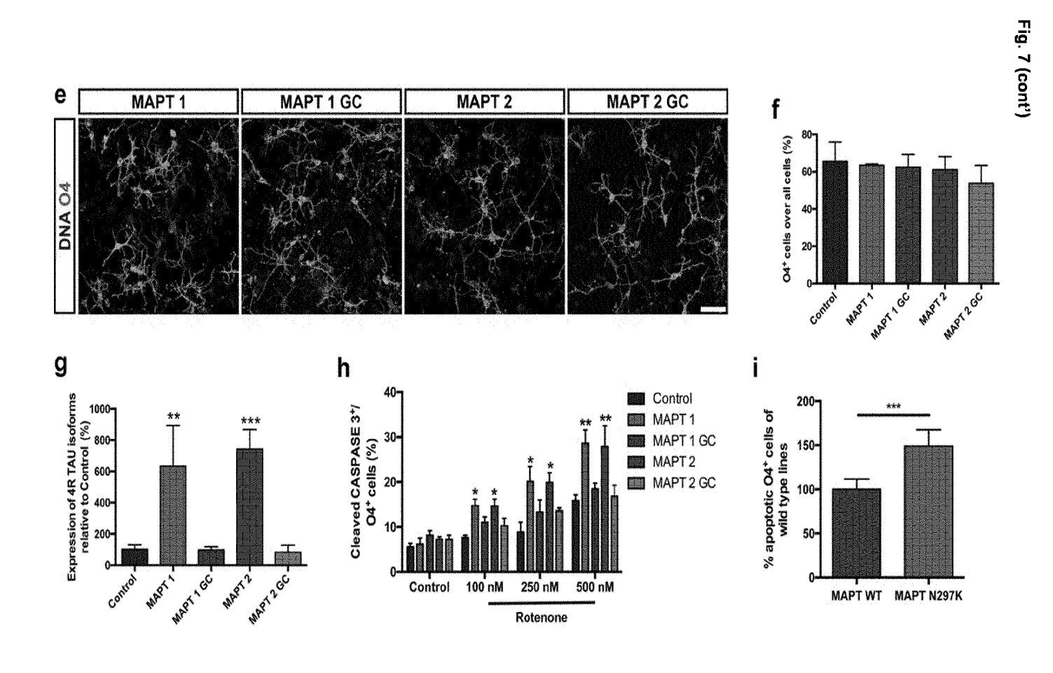

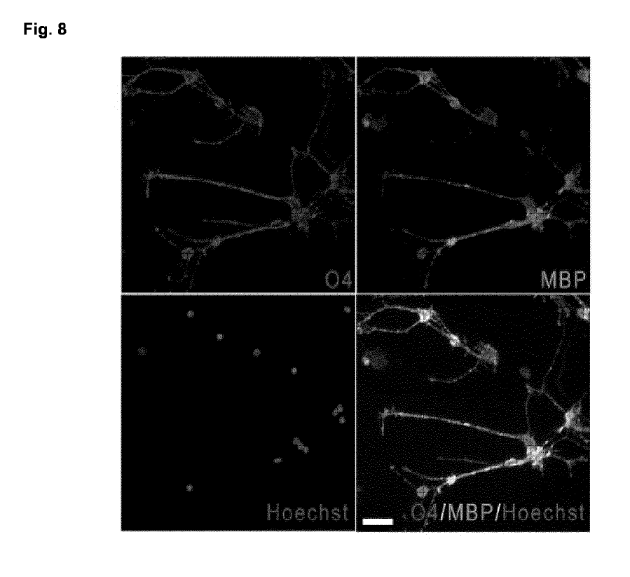

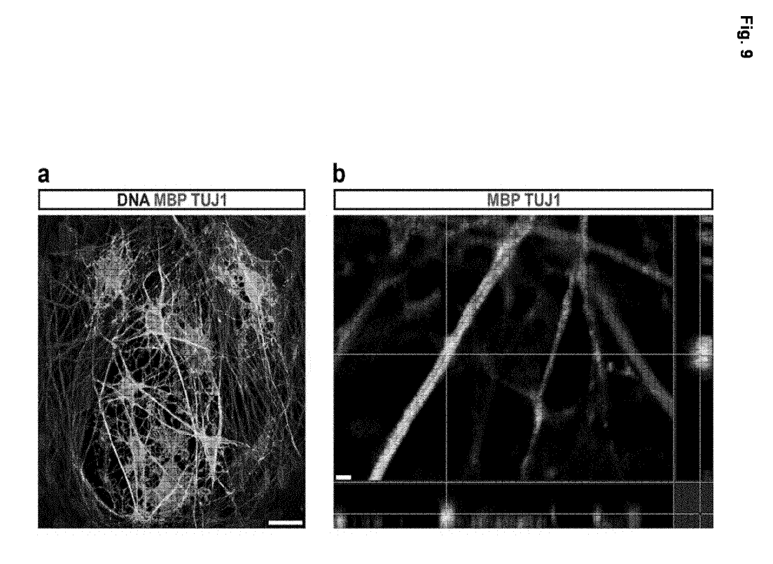

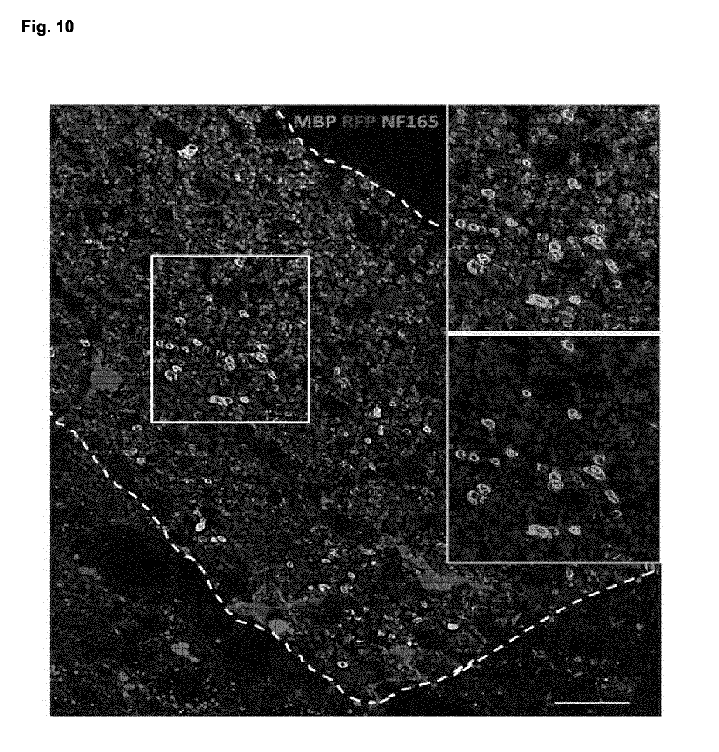

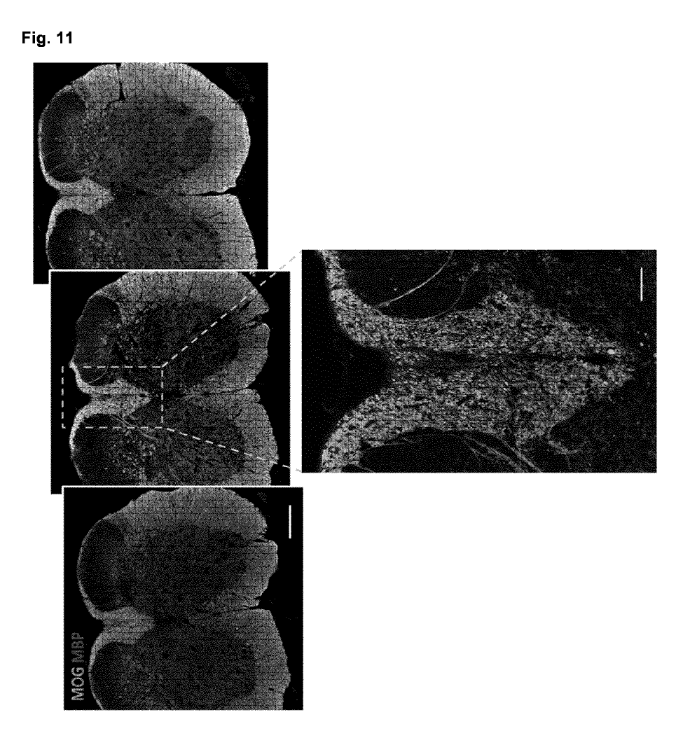

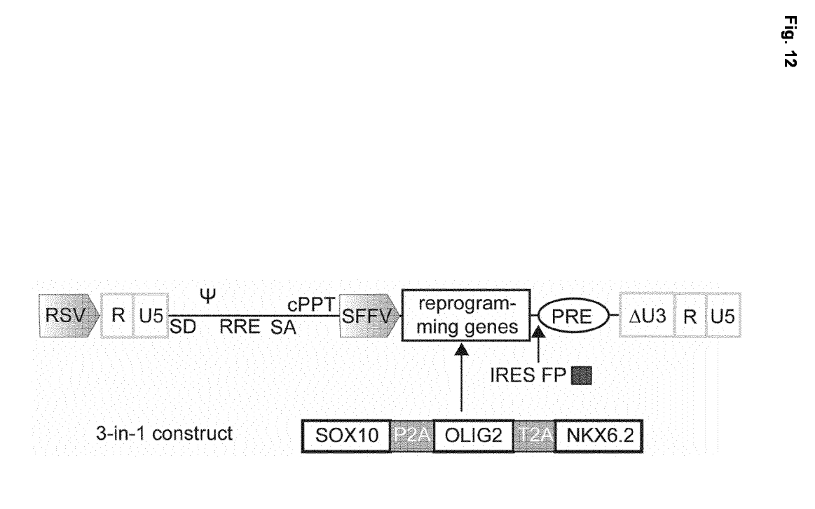

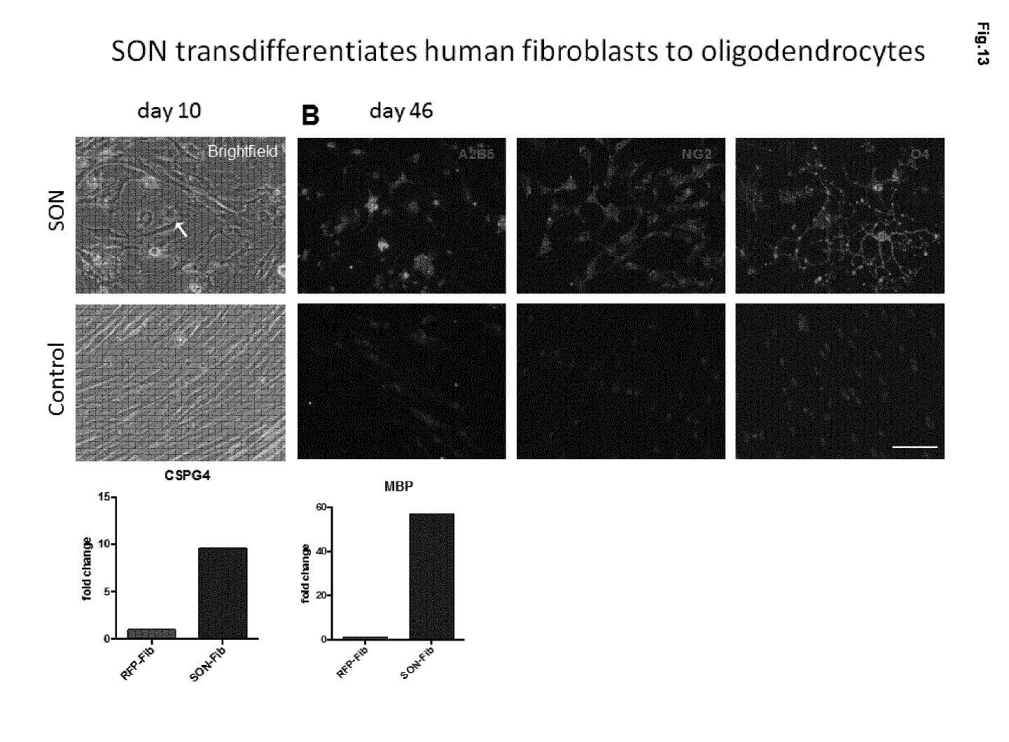

Human pluripotent stem cells present a valuable source for the generation of myelinogenic OL for research and autologous cell replacement therapies⁹⁻¹². NPC are rapidly and efficiently derived from human pluripotent stem cells, but oligodendroglial specification and differentiation is the rate-limiting step in these protocols. Therefore, we first aimed to identify TFs accelerating the oligodendroglial specification and differentiation from human iPSC-derived NPC. We performed literature data mining and selected a set of seven TFs which are enriched in OL compared to other neural lineages^{2, 14} and are required for oligodendroglial specification^{15, 16}: ASCL1, MYT1, NKX2.2, NKX6.1, NKX6.2, OLIG2 and SOX10. Coding sequences for these proteins as well as red fluorescent protein (RFP) were individually cloned into a doxycycline-inducible lentiviral vector. Human iPSC-derived NPC which can be frozen and cost-efficiently expanded as previously described¹⁷ were transduced with a combination of lentiviruses expressing one of the TF candidates and the reverse tetracycline-controlled transactivator (rtTA). Among all TF candidates, only SOX10 was capable of inducing O4 (9.99±0.81%), a highly specific marker of late stage oligodendroglial progenitor cells (OPC) and OL, after 14 days of exposure (FIG. 1b). Controls, including NPC either uninfected or infected with RFP only, did not yield any O4⁺ cells (FIG. 1a). We subsequently determined the oligodendroglial induction capacity of SOX10 in combination with any of the remaining six TFs. We identified OLIG2 as a factor which substantially increased the SOX10-mediated oligodendroglial lineage commitment (FIG. 1g), whereas ASCL1 and MYT1 significantly decreased the number of O4⁺ cells. Co-expression of ASCL1 with SOX10 led to a more immature morphology indicating an inhibitory influence of ASCL1 on the oligodendroglial maturation (FIG. 1d). The combination of SOX10 and OLIG2 with NKX6.2, a TF associated with oligodendroglial maturation, significantly increased further the portion of O4⁺ cells (FIGS. 1e and h). Additionally, we observed the emergence of O4⁺ OL with a more mature morphology and ramified processes indicating an enhanced maturation mediated by NKX6.2 (FIG. 1f). Thus, we concluded that the ectopic expression of SOX10, OLIG2 and NKX6.2 (subsequently referred to as SON) was the best combination of TFs inducing OL from iPSC-derived NPC.

### EXAMPLE 3

### Oligodendroglial Induction is Rapid and Efficient

To further enhance the generation of human iOL, we generated a polycistronic lentiviral expression vector containing SON and RFP as a reporter gene under control of the retroviral spleen focus forming virus (SFFV) promoter (FIG. 2a). iPSC-derived NPC¹³ were infected with SON/RFP expressing lentivirus (FIG. 2b). After induction of SON, a two-step differentiation protocol was sufficient to derive increasing numbers of iOL over 28 days (FIG. 2b). To ensure the reproducibility of our protocol, all experiments were performed with four independent NPC lines derived from three different iPSC lines and one embryonic stem cell (ESC) line. All NPC lines homogeneously expressed the neural stem cell marker SOX1 and NESTIN (FIG. 2c). Seven days after SON induction, OL cultures comprised O4⁺ cells with an immature morphology together with NG2-expressing progenitor cells (FIG. 2d). Further differentiation led to the development of a mature morphology including ramified processes and expression of mature oligodendroglial markers like GALC and MBP by day 28 and 35 respectively (FIGS. 2e and f).

To assess the kinetics and efficiency of SON-mediated oligodendroglial lineage specification, we conducted weekly flow cytometry analyses of the O4 epitope expression during differentiation (FIG. 2g). As a control, NPC were infected with RFP expressing lentivirus. All NPC lines tested were found to perform similarly with respect to OL generation starting from 8.7±3.0% O4⁺ cells at day seven to 65.5±11.1% O4⁺ cells by day 28 (FIG. 2h). On the contrary, only 1.4±0.5% O4⁺ cells were identified in RFP transduced cell cultures (FIG. 2h). The protocol was highly efficient and reproducible among all cell lines illustrated by the quantification of O4⁺ cells at day 28 ranging from 62.1±9.5% (ESC-NPC) to 79.0±14.8% (iPSC-NPC-3) (FIG. 2i). Furthermore, flow cytometry analyses exhibited the presence of an O4⁺/RFP⁻ cell population in SON-transduced cultures (FIG. 2g) which comprised up to 50% of the O4⁺ cell population (data not shown) suggesting transgene silencing in a subset of iOL. Immunocytochemical (ICC) analysis between O4⁺/RFP⁺ and O4⁺/RFP⁻ cells revealed no morphological differences at day 28 indicating that iOL become independent from transgene expression during differentiation (FIG. 2m).

Next, we determined the influence of SON overexpression on the lineage commitment of NPC. ICC analysis of SON infected NPC cultures compared to RFP infected control cultures at day 28 revealed a decreased number of SOX1⁺ NPC (FIG. 2j) and a significant switch from neuronal to oligodendroglial cell fate (FIG. 2k). In contrast, the astroglial lineage commitment was not affected (FIG. 2l).

We then asked whether the SON-expressing cells expand as suggested by FACS analysis (FIG. 2g). Identification of proliferative cells using KI67 revealed a proliferation rate of 35% among RFP⁺ cells at day 14 which declined to 10% by day 28, illustrating that the transgene-expressing cell population further expanded during differentiation (FIG. 2o). Interestingly, the proliferation capability was retained in 20% of O4⁺ iOL at day 14 (FIG. 2n) and diminished to 5% by day 28 (FIG. 2p).

### EXAMPLE 4

Global Gene Expression Profiling Demonstrates that iOL Resemble Primary Human Adult OL

To further characterize the cellular identity of iOL, we compared the global gene expression profiles of purified O4⁺ iOL with human primary OL (pOL) derived from surgically resected brain samples from adult patients (FIG. 8) as well as with iPSC-derived NPC before induction of SON. As a negative control, we utilized gene expression values of undifferentiated iPSC. The unbiased hierarchical clustering clearly demonstrated that iOL and pOL exhibit highly comparable gene-expression signatures and form a distinct cluster significantly segregating from NPC and iPSC (FIG. 3a). When we compared neural lineage specific gene sets, we identified a strong upregulation of oligodendrocyte-specific genes such as OLIG1, MOG and MBP in iOL compared to NPC whereas NPC-related genes including SOX1, PAX6 and PAX7 were downregulated in iOL (FIGS. 3b and c). Interestingly, iOL also expressed some OPC-specific genes such as PDGFRA and ST8SIA1 indicating a more immature cell identity of iOL compared to pOL (FIG. 3d). To further analyze the influence of ectopic SON expression on the oligodendroglial lineage commitment of NPC, we determined differentially expressed genes in iOL compared to the original NPC population. This analysis revealed 755 commonly up- and 955 commonly down-regulated genes among all iOL cell lines (FIGS. 3e and f). Gene ontology (GO) terms associated with upregulated genes in iOL include categories such as “cell adhesion”, “myelin sheath”, “axon ensheathment”, “myelin” and “regulation of action potential”. Conversely, GO terms associated with downregulated genes include categories such as “cell cycle”, “DNA replication”, “mitosis” and “nucleoplasm” (Tables 3 and 4).

TABLE 3
Gene ontology analysis performed for upregulated genes in iOL compared
to iPSC-derived NPC

| Upregulated - GO Term | Genes | P value |
|---|---|---|
| Cell adhesion | 66 | 4.1E−12 |
| Regulation of action potential | 13 | 1.1E−6 |
| Lipoprotein | 41 | 1.3E−5 |
| Myelin sheath | 6 | 7.9E−5 |
| Axon ensheatment | 8 | 3.0E−4 |
| Ensheatment of neurons | 8 | 3.1E−4 |
| Myelination | 6 | 6.3E−3 |

TABLE 4
Gene ontology analysis performed for downregulated genes in iOL
compared to iPSC-derived NPC

| Downregulated - GO Term | Genes | P value |
|---|---|---|
| DNA replication | 62 | 2.9E−32 |
| Nucleoplasm | 124 | 2.2E−27 |
| Cell cycle | 113 | 1.2E−25 |
| DNA repair | 60 | 1.6E−20 |
| Mitosis | 51 | 2.7E−19 |

These results indicate that ectopic expression of SON induces an oligodendroglial gene-expression profile comparable to native human adult OL.

### EXAMPLE 5

iOL Differentiate into Mature MBP-Expressing OL In Vitro and Produce Myelin-Like Sheaths

Next, we assessed the terminal differentiation potential of iOL in vitro. At day 35, iOL cultures contained many highly branched O4⁺ cells (FIG. 4a) as well as mature OL expressing CNP (FIG. 4b) and MAG (FIG. 4c). Additionally, 30.37±7.87% of O4⁺ cells differentiated into mature MBP⁺ iOL with myelin-like sheaths (FIGS. 4d and e). To evaluate the myelinogenic capability of iOL in vitro, we purified O4⁺ iOL using magnetic cell separation (MACS) at day 21 and cultured them for 14 days on 3D cell culture surfaces with aligned nanofibers. ICC analysis of mature MBP⁺ iOL in these cultures revealed the extension of multiple processes along the nanofibers with some of these extensions wrapping around the nanofibers (FIG. 4f). Evidence for ensheathment of axons in vitro was evaluated in co-cultures of O4⁺ iOL with iPSC-derived neurons. After three weeks, the cultures exhibited myelin-like sheaths surrounding the axons, identified by confocal analysis of MBP and TUJ1 expression (FIG. 9a). 3D reconstruction of confocal optical sections in high magnification showed co-labeling of neuronal processes (TUJ1) with MBP (FIG. 4g-h), which was further evaluated by orthogonal projections clearly displaying the formation of MBP⁺ structures around neuronal processes (FIG. 9b). Control cultures completely lacked these MBP⁺ structures. These data clearly illustrate the capability of iOL to mature into MBP⁺ OL and to ensheath neuronal processes in vitro.

### EXAMPLE 6

### iOL Myelinate the Developing Brain and Remyelinate the Demyelinated Spinal Cord of Dysmyelinating Mice

The differentiation of iOL into myelin forming OL was further validated by grafting day 14 MACS-purified O4⁺ iOL in the immune- and MBP-deficient Shi/Shi Rag2^{-/-} mouse CNS. To address developmental myelination, cells were grafted bi-laterally and rostrally to the corpus callosum of newborn mice. Analysis of sagittal sections 16 weeks post grafting (wpg) indicated the presence of numerous areas with MBP⁺ myelin as well as RFP⁺ and human nucleic positive (STEM101⁺) cells (FIGS. 5a and b). Higher magnification using confocal microscopy showed that iOL extended processes frequently to MBP⁺ myelin, thus validating the donor origin of the myelin (FIG. 5c). MBP⁺ myelin generated by grafted iOL wrapped around host axons and was associated with the paranodal marker CASPR (FIGS. 5d and e), demonstrating functionality of human cell-derived myelin. Some of the animals were also used for ultrastructural analysis of myelin compaction. In control Shi/Shi Rag2^{-/-} mice myelin sheaths were thin and non-compacted. In mice that received iOL, numerous normal compacted myelin sheaths with alternating major dense lines and intermediate lines were observed (FIG. 5f), validating unambiguously that iOL have the capacity to differentiate into functional myelin-forming cells in vivo.

To address the ability of iOL to remyelinate demyelinated axons, iOL were grafted into the dorsal funiculus in the spinal cord of adult Shi/Shi Rag2^{-/-} mice that had been injected with lysophosphatidylcholines (LPC) to induce demyelination. 12 wpg immunolabeling of serial cross sections for STEM101 and RFP together with MBP revealed widespread MBP⁺ donut-like myelin structures suggesting that grafted iOL not only colonized and remyelinated the lesion site, but also myelinated the entire neuraxis, including ventral and dorsal white and grey matter (FIG. 6a-c). The extent of human-derived myelination in the spinal cord of Shi/Shi Rag2^{-/-} mice was evaluated by immunolabeling of MBP and MOG and further confirmed the widespread, integrated and high amount of human cell-derived myelin (FIG. 11). Higher magnification showed that processes extended by iOL were frequently connected to MBP⁺ myelin, thus validating the exogenous source of the myelin (FIGS. 6d and f). While most NF⁺ axons were surrounded by RFP⁺ processes, fewer of them co-expressed MBP indicating that myelination was still ongoing (FIG. 6e, FIG. 10). MBP⁺ myelin structures were often co-labeled for the paranodal protein CASPR as viewed on longitudinal and coronal sections (FIGS. 6g and h) indicating the formation of nodes of Ranvier and suggesting that the iOL-derived newly-formed myelin was functional in the adult demyelinated spinal cord.

### EXAMPLE 7

### iOL Facilitate the Identification of Compounds Promoting Oligodendroglial Differentiation and can be Used for Disease Modeling

Identification of drugs inducing remyelination via promotion of oligodendroglial differentiation presents a promising approach for the treatment of demyelinating disorders like MS. Thus, we assessed whether iOL can be utilized to identify compounds promoting oligodendroglial differentiation. We selected six drug candidates (miconazole, clobetasol, benztropine, indometacin, clemastine and oxybutynin) which have been previously described to promote differentiation or myelination of rodent OL^{1, 18-20.} iOL cultures were treated with either vehicle (0.01% (v/v) DMSO) as a negative control, thyroid hormone (T3) as a positive control, or the drug candidate dissolved in DMSO at three different concentrations (0.5 μM, 1 μM, 5 μM) (FIG. 7a-d). In DMSO-treated control cultures, 14.01±2.89% O4⁺ iOL were observed in minimum differentiation medium (DM) after 21 days of culture, whereas addition of T3 resulted in the doubling of O4⁺ cells (28.25±3.47%). Several drug candidates performed as well as T3 and demonstrated a dose-dependent increase of O4⁺ cells (FIG. 7b). Interestingly, clemastine and oxybutynin failed to promote the formation of O4⁺ iOL. Furthermore, we observed a toxic influence of miconazole, benztropine and clemastine on iOL at higher concentrations. Quantification of mature MBP⁺ iOL revealed a fourfold increase in the presence of T3 compared to DMSO control (FIG. 7d). The effect was most striking with 1 μM miconazole, inducing an almost tenfold increase of MBP⁺ cells compared to DMSO control cultures. Clobetasol and benztropine enhanced the formation of MBP⁺ mature iOL comparable to T3-treated cultures. These data demonstrate that iOL can be used to identify compounds that promote differentiation into O4⁺ as well as maturation into MBP⁺ OL.

Next, we wanted to determine whether iOL can be used for disease modeling in vitro. The microtubule associated protein TAU (MAPT) is developmentally expressed in OL^{21, 22} and mutations in MAPT have been associated with frontotemporal dementia with Parkinsonism linked to chromosome 17 (FTDP-17), a disease also characterized by pathological changes in white matter²³⁻²⁵. Therefore, we generated iOL from two iPSC clones from one patient carrying the N279K MAPT mutation associated with FTDP-17¹⁷ and compared these to their isogenic controls. Additionally, we included another independent control iPSC line.

After 28 days of differentiation, O4⁺ iOL harboring the N279K mutation (MAPT-OL) were morphologically indistinguishable from their gene corrected control cell lines (MAPT-GC-OL) (FIG. 7e) and featured similar differentiation efficiencies among all cell lines included (FIG. 7f). We next set out to investigate whether the N279K MAPT mutation induces an altered expression of TAU isoforms in iOL. We purified O4⁺ iOL using FACS before RNA sample preparation. Analysis of TAU expression revealed mutation-specific significantly higher levels of 4R TAU compared to MAPT-GC-OL (FIG. 7g) which is in line with observations in iPSC-derived neurons and brains of FTDP-17 patients harboring this mutation^{17, 26}. FTDP-17 patients display widespread neurodegeneration due to increased cellular vulnerability. Therefore, we investigated whether MAPT-OL are more susceptible to oxidative stress induced by rotenone, an inhibitor of the mitochondrial complex I. Exposure of MAPT- and MAPT-GC-OL for 48 h to rotenone increased MAPT-OL vulnerability to oxidative stress identified by an increased number of cleaved CASPASE-3⁺ iOL in MAPT-cultures (FIG. 7h). This effect was obvious in all tested concentrations of rotenone (100, 250 and 500 nM) leading to an average increase of cell death of 48.9±18.7% in MAPT-OL (FIG. 7i).

### EXAMPLE 8

### SON Transdifferentiates Human Fibtoblasts to Oligodendrocytes

Human dermal fibroblasts were either transduced with SON or RFP expressing lentivirus. 48 h post transduction, culture medium was changed to oligodendroglial differentiation medium. (as described herein for iOLs). Immunocytochemical analysis and RNA samples were obtained at day 46 of differentiation. Results are shown in FIG. 13.

### REFERENCE LIST

1. Najm, F. J. et al. Drug-based modulation of endogenous stem cells promotes functional remyelination in vivo. Nature 522, 216-220 (2015).
2. Yang, N. et al. Generation of oligodendroglial cells by direct lineage conversion. Nat. Biotechnol. 31, 434-439 (2013).
3. Goldman, S. A., Nedergaard, M., & Windrem, M. S. Glial progenitor cell-based treatment and modeling of neurological disease. Science 338, 491-495 (2012).
4. Franklin, R. J., Ffrench-Constant, C., Edgar, J. M., & Smith, K. J. Neuroprotection and repair in multiple sclerosis. Nat. Rev. Neurol. 8, 624-634 (2012).
5. Sreedharan, J. & Brown, R. H., Jr. Amyotrophic lateral sclerosis: Problems and prospects. Ann. Neurol. 74, 309-316 (2013).
6. Kim, J. B. et al. Oct4-induced oligodendrocyte progenitor cells enhance functional recovery in spinal cord injury model. EMBO J. 34, 2971-2983 (2015).
7. Tallantyre, E. C. et al. Clinico-pathological evidence that axonal loss underlies disability in progressive multiple sclerosis. Mult. Scler. 16, 406-411 (2010).
8. Atkins, H. & Freedman, M. Immune ablation followed by autologous hematopoietic stem cell transplantation for the treatment of poor prognosis multiple sclerosis. Methods Mol. Biol. 549, 231-246 (2009).
9. Douvaras, P. et al. Efficient generation of myelinating oligodendrocytes from primary progressive multiple sclerosis patients by induced pluripotent stem cells. Stem Cell Reports. 3, 250-259 (2014).
10. Stacpoole, S. R. et al. High yields of oligodendrocyte lineage cells from human embryonic stem cells at physiological oxygen tensions for evaluation of translational biology. Stem Cell Reports. 1, 437-450 (2013).
11. Wang, S. et al. Human iPSC-derived oligodendrocyte progenitor cells can myelinate and rescue a mouse model of congenital hypomyelination. Cell Stem Cell 12, 252-264 (2013).
12. Piao, J. et al. Human embryonic stem cell-derived oligodendrocyte progenitors remyelinate the brain and rescue behavioral deficits following radiation. Cell Stem Cell 16, 198-210 (2015).
13. Reinhardt, P. et al. Derivation and expansion using only small molecules of human neural progenitors for neurodegenerative disease modeling. PLoS. One. 8, e59252 (2013).
14. Najm, F. J. et al. Transcription factor-mediated reprogramming of fibroblasts to expandable, myelinogenic oligodendrocyte progenitor cells. Nat. Biotechnol. 31, 426-433 (2013).
15. Liu, Z. et al. Induction of oligodendrocyte differentiation by Olig2 and Sox10: evidence for reciprocal interactions and dosage-dependent mechanisms. Dev. Biol. 302, 683-693 (2007).
16. Wang, J. et al. Transcription factor induction of human oligodendrocyte progenitor fate and differentiation. Proc. Natl. Acad. Sci. U.S.A. 111, E2885-E2894 (2014).
17. Ehrlich, M. et al. Distinct Neurodegenerative Changes in an Induced Pluripotent Stem Cell Model of Frontotemporal Dementia Linked to Mutant TAU Protein. Stem Cell Reports. 5, 83-96 (2015).
18. Preisner, A. et al. Non-steroidal anti-inflammatory drug indometacin enhances endogenous remyelination. Acta Neuropathol. 130, 247-261 (2015).
19. Mei, F. et al. Micropillar arrays as a high-throughput screening platform for therapeutics in multiple sclerosis. Nat. Med. 20, 954-960 (2014).
20. Deshmukh, V. A. et al. A regenerative approach to the treatment of multiple sclerosis. Nature 502, 327-332 (2013).
21. LoPresti, P. Regulation and differential expression of tau mRNA isoforms as oligodendrocytes mature in vivo: implications for myelination. Glia 37, 250-257 (2002).
22. Gorath, M., Stahnke, T., Mronga, T., Goldbaum, O., & Richter-Landsberg, C. Developmental changes of Tau protein and mRNA in cultured rat brain oligodendrocytes. Glia 36, 89-101 (2001).
23. Lam, B. Y., Halliday, G. M., Irish, M., Hodges, J. R., & Piguet, O. Longitudinal white matter changes in frontotemporal dementia subtypes. Hum. Brain Mapp. 35, 3547-3557 (2014).
24. Kovacs, G. G. et al. White matter tauopathy with globular glial inclusions: a distinct sporadic frontotemporal lobar degeneration. J. Neuropathol. Exp. Neurol. 67, 963-975 (2008).
25. Lu, P. H. et al. Regional differences in white matter breakdown between frontotemporal dementia and early-onset Alzheimer's disease. J. Alzheimers. Dis. 39, 261-269 (2014).
26. Goedert, M. & Spillantini, M. G. Tau mutations in frontotemporal dementia FTDP-17 and their relevance for Alzheimer's disease. Biochim. Biophys. Acta 1502, 110-121 (2000).
27. Sugimori, M. et al. Ascii is required for oligodendrocyte development in the spinal cord. Development 135, 1271-1281 (2008).
28. Pozniak, C. D. et al. Sox10 directs neural stem cells toward the oligodendrocyte lineage by decreasing Suppressor of Fused expression. Proc. Natl. Acad. Sci. U.S.A. 107, 21795-21800 (2010).
29. Lu, Q. R. et al. Sonic hedgehog—regulated oligodendrocyte lineage genes encoding bHLH proteins in the mammalian central nervous system. Neuron 25, 317-329 (2000).
30. Jessberger, S., Toni, N., Clemenson, G. D., Jr., Ray, J., & Gage, F. H. Directed differentiation of hippocampal stem/progenitor cells in the adult brain. Nat. Neurosci. 11, 888-893 (2008).
31. Maire, C. L. et al. Directing human neural stem/precursor cells into oligodendrocytes by overexpression of Olig2 transcription factor. J. Neurosci. Res. 87, 3438-3446 (2009).
32. Lee, S. et al. A culture system to study oligodendrocyte myelination processes using engineered nanofibers. Nat. Methods 9, 917-922 (2012).
33. Bechler, M. E., Byrne, L., & Ffrench-Constant, C. CNS Myelin Sheath Lengths Are an Intrinsic Property of Oligodendrocytes. Curr. Biol. 25, 2411-2416 (2015).
34. Windrem, M. S. et al. Fetal and adult human oligodendrocyte progenitor cell isolates myelinate the congenitally dysmyelinated brain. Nat. Med. 10, 93-97 (2004).
35. Windrem, M. S. et al. Neonatal chimerization with human glial progenitor cells can both remyelinate and rescue the otherwise lethally hypomyelinated shiverer mouse. Cell Stem Cell 2, 553-565 (2008).
36. Buchet, D., Garcia, C., Deboux, C., Nait-Oumesmar, B., & Baron-Van, E. A. Human neural progenitors from different foetal forebrain regions remyelinate the adult mouse spinal cord. Brain 134, 1168-1183 (2011).
37. Mozafari, S. et al. Skin-derived neural precursors competitively generate functional myelin in adult demyelinated mice. J. Clin. Invest 125, 3642-3656 (2015).
38. Ghetti, B. et al. Invited review: Frontotemporal dementia caused by microtubule-associated protein tau gene (MAPT) mutations: a chameleon for neuropathology and neuroimaging. Neuropathol. Appl. Neurobiol. 41, 24-46 (2015).
39. Seiberlich, V. et al. Downregulation of the microtubule associated protein tau impairs process outgrowth and myelin basic protein mRNA transport in oligodendrocytes. Glia 63, 1621-1635 (2015).
40. Klein, C. et al. Process outgrowth of oligodendrocytes is promoted by interaction of fyn kinase with the cytoskeletal protein tau. J Neurosci 22, 698-707 (2002).
41. Ahfeldt, T. et al. Programming human pluripotent stem cells into white and brown adipocytes. Nat. Cell Biol. 14, 209-219 (2012).
42. Warlich, E. et al. Lentiviral vector design and imaging approaches to visualize the early stages of cellular reprogramming. Mol. Ther. 19, 782-789 (2011).
43. Cui, Q. L. et al. Response of Human Oligodendrocyte Progenitors to Growth Factors and Axon Signals. J Neuropathol. Exp. Neurol (2010).
44. Carvalho, B. S. & Irizarry, R. A. A framework for oligonucleotide microarray preprocessing. Bioinformatics. 26, 2363-2367 (2010).
45. Irizarry, R. A. et al. Exploration, normalization, and summaries of high density oligonucleotide array probe level data. Biostatistics. 4, 249-264 (2003).
46. Marteyn, A. et al. Modulation of the Innate Immune Response by Human Neural Precursors Prevails Over Oligodendrocyte Progenitor Remyelination to Rescue a Severe Model Of Pelizaeus-Merzbacher Disease. Stem cells (2015).
47. Geerts. Of mice and men: bridging the translational disconnect in CNS drug discovery. CNS Drugs; 23(11):915-26 (2009).
48. Takahashi and Yamanaka. Induction of pluripotent stem cells from mouse embryonic and adult fibroblast cultures by defined factors. Cell 126:663-76 (2006)
49. Takahashi et al. Induction of pluripotent stem cells from adult human fibroblasts by defined factors. Cell; 131(5):861-72 (2007).
50. Reinhardt et al. Derivation and expansion using only small molecules of human neural progenitors for neurodegenerative disease modeling. PLoS One 8:e59252; (2013).
51. Major et al. Transgene excision has no impact on in vivo integration of human iPS derived neural precursors. PLoS One; 6(9):e24687; (2011).
52. Bradl et al. Oligodendrocytes: biology and pathology. Acta Neuropathol 119:37-53 (2010).
53. Dainiak et al. Adv Biochem Eng Biotechnol. 2007; 106:1-18
54. Thompson Nucl. Acids Res. 2 (1994), 4673-4680
55. Brutlag Comp. App. Biosci. 6 (1990), 237-245
56. Altschul, Nucl. Acids Res. 25 (1997), 3389-3402
57. Altschul, J. Mol. Evol. 36 (1993), 290-300
58. Altschul, J. Mol. Biol. 215 (1990), 403-410

1. A method of generating oligodendroglial lineage cells, the method comprising the steps of:
(a) providing human cells selected from the group consisting of neural progenitor cells (NPCs), pluripotent stem cells (PSCs), induced pluripotent stem cells (iPSCs) and fibroblasts;
(b) inducing and/or increasing expression of the transcription factor SOX10, optionally in combination with OLIG2 and/or NKX6.2 in the cells;
(c) culturing the cells; thereby generating the oligodendroglial lineage cells.

2. The method of claim 1, wherein the oligodendroglial lineage cells express one or more markers selected from the group consisting of PDGFRA, ST8SIA1, NG2, O4, GALC, O1, PLP, MBP, CNP, MAG, OLIG1, MOG, and a combination thereof.

3. The method of any one of the preceding claims, wherein the NPCs are derived from PSCs or iPSCs.

4. The method of any one of the preceding claims, wherein the expression of one or more of the transcription factors SOX10, OLIG2 and NKX6.2 in step (b) is increased compared to endogenous expression of the corresponding transcription factors.

5. The method of any one of the preceding claims, wherein the expression of one or more of the transcription factors SOX10, OLIG2 and NKX6.2 is an ectopic expression.

6. The method of any one of the preceding claims, wherein one or more nucleic acid(s) encoding one or more of the transcription factors SOX10, OLIG2 and NKX6.2 is/are introduced in the cells of step (a).

7. The method of any one of the preceding claims, wherein, in step (c), the cells are cultured for a pre-determined amount of time following inducing and/or increasing expression, e.g., for at least 7, 14, 21, 28 or 35 days following inducing and/or increasing expression.

8. The method of any one of the preceding claims, wherein, after culturing the cells in step (c) for 7 days following inducing and/or increasing expression, at least 5%, preferably at least 6%, more preferably at least 7%, still more preferably at least 8% of the cells are O4⁺ oligodendroglial lineage cells.

9. The method of any one of the preceding claims, wherein, after culturing the cells in step (c) for 14 days following inducing and/or increasing expression, at least 15%, preferably at least 16%, more preferably at least 17%, still more preferably at least 18% of the cells are O4⁺ oligodendroglial lineage cells.

10. The method of any one of the preceding claims, wherein, after culturing the cells in step (c) for 21 days following inducing and/or increasing expression, at least 30%, preferably at least 33%, more preferably at least 36%, still more preferably at least 39% of the cells are O4⁺ oligodendroglial lineage cells.

11. The method of any one of the preceding claims, wherein, after culturing the cells in step (c) for 28 days following inducing and/or increasing expression, at least 55%, preferably at least 59%, more preferably at least 63%, still more preferably at least 67% of the cells are O4⁺ oligodendroglial lineage cells.

12. The method of any one of the preceding claims, wherein, after culturing the cells in step (c) for about 35 days following inducing and/or increasing expression, at least 20%, preferably at least 25%, more preferably at least 30%, still more preferably at least 35% of O4⁺ oligodendroglial lineage cells are also MBP⁺.

13. An oligodendroglial lineage cell obtainable by the method of any one of the preceding claims, preferably wherein the cell is O4⁺ and/or MBP⁺.

14. A recombinant vector comprising a nucleotide sequence encoding SOX10, OLIG2 and NKX6.2, wherein the vector is a non-viral vector or a viral vector, e.g. a retroviral vector, preferably a lentiviral vector.

15. A human NPC, PSC, iPSC or fibroblast comprising one or more exogenous nucleic acid(s) encoding at least one or more of SOX10, OLIG2 and NKX6.2, preferably wherein the one or more nucleic acid(s) encode(s) SOX10 and OLIG2 and optionally NKX6.2.

16. A method of screening for a compound promoting oligodendroglial differentiation and/or maturation, the method comprising the steps of:
(a) providing human cells selected from the group consisting of NPCs, PSCs, iPSCs and fibroblasts or providing cells according to claim 15;
(b) inducing and/or increasing expression of the transcription factor SOX10, optionally in combination with OLIG2 and/or NKX6.2 in the cells;
(c) culturing the cells for a pre-determined amount of time following inducing and/or increasing expression, wherein a first sample of the cells is cultured in the presence of a compound to be tested and a second sample of the cells is cultured in the absence of the compound;
(d) determining the percentage of cells which are positive for a marker of an oligodendrocyte developmental stage in the first sample and in the second sample;
wherein a higher percentage of cells which are positive for the marker in the first sample than in the second sample indicates that the compound promotes oligodendroglial differentiation and/or maturation.

17. The method of screening of claim 16, wherein the marker is selected from the group consisting of PDGFRA, ST8SIA1, NG2, O4, GALC, O1, PLP, MBP, CNP, MAG, OLIG1, MOG, and a combination thereof.

18. A use of oligodendroglial lineage cells obtainable by the method of any one of claims 1-12 or of a cell of claim 15 in a screening method, preferably wherein the screening method is a high throughput screening, or in expression profiling or in disease modeling.

19. A pharmaceutical composition comprising cells obtainable by the method of any one of claims 1-12 and/or comprising cells of claim 15.

20. The pharmaceutical composition of claim 19, the cell obtainable by the method of any one of claims 1-12, or the cell of any one of claim 15 for use as a medicament.


SEQUENCE LISTING (9 sequences)

SEQ ID NO: 1 - DNA, 25 bp, artificial
  oligonucleotide for PCR (hGAPDH)
ctggtaaagtggatattgttgccat

SEQ ID NO: 2 - DNA, 26 bp, artificial
  oligonucleotide for PCT (hGAPDH)
tggaatcatattggaacatgtaaacc

SEQ ID NO: 3 - DNA, 22 bp, artificial
  oligonucleotide for PCR (hMAPT)
ctcgcatggtcagtaaaagcaa

SEQ ID NO: 4 - DNA, 22 bp, artificial
  oligonucleotide for PCR (hMAPT)
gggtttttgctggaatcctggt

SEQ ID NO: 5 - DNA, 20 bp, artificial
  oligonucleotide for PCR (hMAPT exon10)
ccaagtgtggctcaaaggat

SEQ ID NO: 6 - DNA, 20 bp, artificial
  oligonucleotide for PCR (hMAPT exon 12)
cccaatcttcgactggactc

SEQ ID NO: 7 - protein, 466 aa, Homo sapiens
MAEEQDLSEVELSPVGSEEPRCLSPGSAPSLGPDGGGGGSGLRASPGPGELGKVKKEQQDGEADDDKFPVCIREAVSQVLSGYDWTLVPMPVRVNGASKSKPHVKRPMNAFMVWAQAARRKLADQYPHLHNAELSKTLGKLWRLLNESDKRPFIEEAERLRMQHKKDHPDYKYQPRRRKNGKAAQGEAECPGGEAEQGGTAAIQAHYKSAHLDHRHPGEGSPMSDGNPEHPSGQSHGPPTPPTTPKTELQSGKADPKRDGRSMGEGGKPHIDFGNVDIGEISHEVMSNMETFDVAELDQYLPPNGHPGHVSSYSAAGYGLGSALAVASGHSAWISKPPGVALPTVSPPGVDAKAQVKTETAGPQGPPHYTDQPSTSQIAYTSLSLPHYGSAFPSISRPQFDYSDHQPSGPYYGHSGQASGLYSAFSYMGPSQRPLYTAISDPSPSGPQSHSPTHWEQPVYTTLSRP

SEQ ID NO: 8 - protein, 323 aa, Homo sapiens
MDSDASLVSSRPSSPEPDDLFLPARSKGSSGSAFTGGTVSSSTPSDCPPELSAELRGAMGSAGAHPGDKLGGSGFKSSSSSTSSSTSSAAASSTKKDKKQMTEPELQQLRLKINSRERKRMHDLNIAMDGLREVMPYAHGPSVRKLSKIATLLLARNYILMLTNSLEEMKRLVSEIYGGHHAGFHPSACGGLAHSAPLPAATAHPAAAAHAAHHPAVHHPILPPAAAAAAAAAAAAAVSSASLPGSGLPSVGSIRPPHGLLKSPSAAAAAPLGGGGGGSGASGGFQHWGGMPCPCSMCQVPPPHHHVSAMGAGSLPRLTSDAK

SEQ ID NO: 9 - protein, 277 aa, Homo sapiens
MDTNRPGAFVLSSAPLAALHNMAEMKTSLFPYALQGPAGFKAPALGGLGAQLPLGTPHGISDILGRPVGAAGGGLLGGLPRLNGLASSAGVYFGPAAAVARGYPKPLAELPGRPPIFWPGVVQGAPWRDPRLAGPAPAGGVLDKDGKKKHSRPTFSGQQIFALEKTFEQTKYLAGPERARLAYSLGMTESQVKVWFQNRRTKWRKRHAAEMASAKKKQDSDAEKLKVGGSDAEDDDEYNRPLDPNSDDEKITRLLKKHKPSNLALVSPCGGGAGDAL